(19) United States
(12) Patent Application Publication
(10) Pub. No.: US 2020/0276249 A1
(43) Pub. Date: Sep. 3, 2020

(54) MANIPULATION OF TRYPTAMINE METABOLISM

(71) Applicant: Seres Therapeutics, Inc., Cambridge, MA (US)

(72) Inventors: Edward J. O'BRIEN, Arlington, MA (US); Asuncion MARTINEZ, Arlington, MA (US)

(73) Assignee: Seres Therapeutics, Inc., Cambridge, MA (US)

(21) Appl. No.: 16/753,545

(22) PCT Filed: Oct. 3, 2018
(86) PCT No.: PCT/US2018/054252

§ 371 Date: Apr. 3, 2020

Related U.S. Application Data
(60) Provisional application No. 62/567,415, filed on Oct. 3, 2017.

(51) Int. Cl. A61K 35/742

(52) U.S. Cl. CPC A61K 35/742; A61K 2035/115

(57) ABSTRACT

Compositions and methods for altering tryptamine levels in a subject are provided. In general, the compositions comprise microorganisms.

### FIELD OF THE INVENTION

The invention relates to manipulation of tryptophan metabolism using microbial compositions.

### BACKGROUND

Besides its use in protein synthesis, tryptophan is important in a number of pathways leading to the production of, for example, serotonin (5-hydroxytryptamine), melatonin, kynurenines, and tryptamine. Tryptophan and its metabolites can affect, for example, immunosuppression, immune function, cancer, inflammatory disease, epithelial barrier function, gut motility, neurologic function, and infection.

Certain tryptophan pathway products have been shown to function as aryl hydrocarbon receptor (Ahr) agonists. The metabolites include, for example, indole-3 aldehyde, indole-3 acetate, indole-3 propionic acid, indole, indole-3 acetaldehyde, indoleacrylic acid, indole-3 acetonitrile, 6-formylindolo[3,2-b]carbazole (FICZ), and tryptamine. Ahr plays a role in controlling the differentiation and activity of specific T-cell subpopulations. It reportedly can influence adaptive immune responses through its effects on both T cells and antigen presenting cells (APCs). Ahr is thought to be involved in development and maintenance of CD4+ FoxP3+T regulatory cells (Tregs), as well as FoxP3− IL-10+CD4+ Tr1, and induction of Th17 cells.

Tryptamine is generated by the decarboxylation of tryptophan. Tryptamine can also have effects on the nervous system, for example, it reportedly affects the myenteric plexus which is involved in gastrointestinal mobility (Takaki et al., Neuroscience 16:223-240, 1985).

The gastrointestinal microbiome has been shown to be manipulable, at least in a global manner, by introducing fecal material into a patient, for example, a patient suffering from recurrent C. difficile infection. Therapeutic manipulation of tryptophan-related effects has generally been limited to chemical manipulation of upstream metabolites. Applicants' discoveries and inventions make possible the manipulation of specific functions in a more targeted manner using bacteria derived from the human microbiome.

### SUMMARY

The invention relates to the identification of bacterial species that can affect (e.g., increase) tryptamine and/or 5-hydroxytryptamine (serotonin) levels. Accordingly, the invention relates to compositions including one or more bacterial species that modulate tryptamine and/or 5-hydroxytryptamine levels, as well as methods for modulating tryptamine and/or 5-hydroxytryptamine levels by administering such compositions. In various embodiments, the levels of tryptamine or 5-hydroxytryptamine are altered (e.g., increased) as compared to levels prior to treatment according to a method or using a composition of the invention.

The invention provides methods of altering tryptamine or 5-hydroxytryptamine levels in a subject, the methods including administering a viable population of at least one bacterial species selected from Table 1, 2, 3, 4, 5, or 6 to the subject. In various embodiments, the levels of tryptamine or 5-hydroxytryptamine in the subject are increased. For example, the levels may be increased as compared to before administration of the viable bacterial population noted above.

In some embodiments, the methods include administering a viable population of at least two species of the group consisting of Ruminococcus_gnavus (strain 1), Lachnospiraceae_bacterium_9_1_43BFAA, Eggerthella_unclassified, Ruminococcus_gnavus (strain 2), Clostridium_nexile, Lachnospiraceae_bacterium_6_1_63FAA, and Ruminococcus_torques to the subject.

In some embodiments, the methods include administering a viable population of at least two species of the group consisting of Clostridium ghonii, Flavonifractor plautii, Ruminococcus gnavus Bacteroides ovatus, Bacteroides stercoris, and Clostridium sporogenes to the subject.

In some embodiments, the methods include administering a viable population of at least two species of the group consisting of Lachnospiraceae_bacterium_2_1_58FAA, Clostridium_aldenense_SC114, Clostridium_citroniae, and Clostridium_clostridioforme to the subject.

In some embodiments, the methods include administering a viable population of at least two species of the group consisting of Flavonifractor_plautii, Veillonella_parvula, Blautia_sp_CAG_257_SC146, and Clostridium_bolteae to the subject.

In some embodiments, the methods include administering a viable population of at least two species of the group consisting of Blautia_hansenii, Lachnospiraceae_bacterium_2_1_46FAA, Coprococcus_sp_HPP048, Collinsella_tanakaei, Clostridium_sporogenes, Clostridium_phytofermentans, Clostridium_bifermentans, Staphylococcus_aureus, Lachnospiraceae_bacterium_4_1_37FAA, Clostridium_asparagiforme, Clostridium_lavalense_SC43, and Holdemania_filliformis to the subject.

In various embodiments of the above, the methods include administering a viable population of at least 3, 4, 5, 6, 7, 8, 9, 10, 11, or 12 of the listed species to the subject (with the upper limit being the number of species in the groups noted above).

Furthermore, in some embodiments, the methods include administering a composition containing or consisting of one or more of compositions 1 to 47 of Table 6 to the subject.

In some embodiments, the methods of the invention can be used with a subject having a disease or condition characterized by altered gut motility, platelet aggregation, immune response, cardiac function, or bone development.

In some embodiments, the subject has a disease or condition selected from the group consisting of irritable bowel syndrome, an inflammatory bowel disease (e.g., infectious colitis, ulcerative colitis, Crohn's disease, ischemic colitis, radiation colitis, and microscopic colitis), constipation, depression, anxiety, cardiovascular disease, and osteoporosis.

The invention also provides pharmaceutical formulations including a viable population of at least one bacterial species selected from Table 1, 2, 3, 4, 5, or 6.

In some embodiments, the pharmaceutical formulation includes a viable population of at least two species of the group consisting of Ruminococcus_gnavus (strain 1), Lachnospiraceae_bacterium_9_1_43BFAA, Eggerthella_unclassified, Ruminococcus_gnavus (strain 2), Clostridium_nexile, Lachnospiraceae_bacterium_6_1_63FAA, and Ruminococcus_torques.

In some embodiments, the pharmaceutical formulation includes a viable population of at least two species of the group consisting of Clostridium ghonii, Flavonifractor plautii, Ruminococcus gnavus Bacteroides ovatus, Bacteroides stercoris, and Clostridium sporogenes.

In some embodiments, the pharmaceutical formulation includes a viable population of at least two species of the group consisting of Lachnospiraceae_bacterium_2_1_58FAA, Clostridium_aldenense_SC114, Clostridium_citroniae, and Clostridium_clostridioforme.

In some embodiments, the pharmaceutical formulation includes a viable population of at least two species of the group consisting of Flavonifractor_plautii, Veillonella_parvula, Blautia_sp_CAG_257_SC146, and Clostridium_bolteae.

In some embodiments, the pharmaceutical formulation includes a viable population of at least two species of the group consisting of Blautia_hansenii, Lachnospiraceae_bacterium_2_1_46FAA, Coprococcus_sp_HPP048, Collinsella_tanakaei, Clostridium_sporogenes, Clostridium_phytofermentans, Clostridium_bifermentans, Staphylococcus_aureus, Lachnospiraceae_bacterium_4_1_37FAA, Clostridium_asparagiforme, Clostridium_lavalense_SC43, and Holdemania_filliformis.

In various embodiments of the above, the pharmaceutical formulation includes a viable population of at least 3, 4, 5, 6, 7, 8, 9, 10, 11, or 12 of the listed species (with the upper limit being the number of species in the groups noted above).

Furthermore, in some embodiments, the pharmaceutical formulations include or consist of one or more of compositions 1 to 47 of Table 6.

In various embodiments, the pharmaceutical formulations include a pharmaceutically acceptable excipient, e.g., as described herein.

In some embodiments, the pharmaceutical formulation is in a capsule. For example, the pharmaceutical formulation can, in some embodiments, can be within an enterically coated capsule.

The invention also provides methods of altering tryptamine or 5-hydroxytryptamine levels in a subject, the methods including administering an effective amount of a pharmaceutical formulation as described herein (see, e.g., above) to the subject.

In some embodiments, the levels of tryptamine or 5-hydroxytryptamine in the subject are increased.

In some embodiments, the tryptamine or 5-hydroxytryptamine levels are tryptamine or 5-hydroxytryptamine levels in feces of the subject.

In some embodiments, the tryptamine or 5-hydroxytryptamine levels are tryptamine or 5-hydroxytryptamine levels in blood, serum, plasma, urine, or cerebrospinal fluid (CSF) of the subject.

The invention further provides methods of treating a subject having a disease or condition characterized by the presence of low tryptamine or 5-hydroxytryptamine levels, the methods including administering to a subject diagnosed with or at risk for the disease with a therapeutically effective amount of a pharmaceutical formulation as described herein (see, e.g., above).

In some embodiments, the subject has a disease or condition characterized by altered gut motility, platelet aggregation, immune response, cardiac function, or bone development.

In some embodiments, the subject has a disease or condition selected from the group consisting of irritable bowel syndrome, an inflammatory bowel disease (e.g., infectious colitis, ulcerative colitis, Crohn's disease, ischemic colitis, radiation colitis, and microscopic colitis), constipation, depression, anxiety, cardiovascular disease, and osteoporosis.

The invention also provides methods of increasing the level or activity of regulatory T cells in subject, the methods including administering a pharmaceutical formulation as described herein (see, e.g., above) to the subject.

Additionally, the invention provides methods for restoring or improving gut homeostasis, or for preventing or treating intestinal or colon cancer in a subject, the methods including administering a pharmaceutical formulation as described herein (see, e.g., above) to the subject.

The invention also provides compositions including at least two different bacterial species that, in combination, can increase tryptamine or 5-hydroxytryptamine levels, as compared to the level of tryptamine or 5-hydroxytryptamine produced by each species alone, (i) in the presence of the same level of tryptophan as the combination, (ii) over a specific period of time in the presence of the same level of tryptophan as the combination, (iii) when administered to an in vivo system, and/or (iv) when administered in vitro to a model system.

In some embodiments, the composition is a composition including the bacterial species of a pharmaceutical formulation as described herein (see, e.g., above).

The invention also provides formulations or compositions, as noted above and elsewhere herein, for use in altering the levels of tryptamine or 5-hydroxytryptamine in a subject.

In some embodiments, the subject has a disease or condition characterized by altered gut motility, platelet aggregation, immune response, cardiac function, or bone development.

In some embodiments, the subject has a disease or condition selected from the group consisting of irritable bowel syndrome, an inflammatory bowel disease (infectious colitis, ulcerative colitis, Crohn's disease, ischemic colitis, radiation colitis, and microscopic colitis), constipation, depression, anxiety, cardiovascular disease, and osteoporosis.

In some embodiments, the formulation or composition is for use in increasing the level or activity of regulatory T cells in subject.

In some embodiments, the formulation or composition is for use in restoring gut homeostasis, or for preventing or treating intestinal or colon cancer in a subject.

The invention includes the use of the compositions and formulations as described herein for the purposes expressed in the methods described above and elsewhere herein, as well as for the preparation of medicaments therefor.

The entire disclosure of each patent document and scientific article referred to herein, and those patent documents and scientific articles cited thereby, is expressly incorporated by reference herein for all purposes.

Additional features and advantages of the invention are more particularly described below.

### BRIEF DESCRIPTION OF THE DRAWINGS

FIG. 1 is a graph depicting the results of an analysis of three species associated with the high levels of tryptamine and tryptamine levels in the presence of one, two, or three of the species. Lb=Lachnospiraceae_bacterium_9_1_43BFAA; Rg=Ruminococcus gnavus; Rt=Ruminococcus_torques; 0 indicates species is absent, 1 indicates species is present. Rt only: low tryptamine levels; Rg only: medium tryptamine levels; 2 of 3 species: medium-to-high tryptamine levels; all 3 species: high tryptamine levels; note: only 1 sample has Lb=1, Rg=0, Rt=0, so it is not shown.

FIG. 2 is a graph showing the results of Blastp of tryptophan decarboxylase (SEQ ID NOs: 73 and 75) versus a microbial species pan-genome database.

FIG. 3 is a graph showing the identification of species whose presence in subject fecal samples is predictive of elevated tryptamine levels in species (compared to subjects who lack the species) across two clinical trials. The encircled species indicate those whose presence is independently predictive of higher tryptamine levels across two clinical trials.

FIG. 4 is a set of graphs showing tryptamine-modulating species richness in two clinical trials SERES-101 (related to maintain remission of ulcerative colitis) and SERES-004 (related to preventing recurrence of C. difficile infection).

FIG. 5 is a list of 16S rDNA sequences of selected bacterial OTUs that can be used in the invention (SEQ ID NOs: 1-72), as well as sequences of certain tryptophan decarboxylases (SEQ ID NOs: 73-75).

### DETAILED DESCRIPTION

Applicants have identified bacterial species in the human gastrointestinal (GI) tract that are associated with the presence of tryptamine. The identities of some species are unexpected, for example, in some cases, the species have not been previously identified as participating in regulating tryptamine. Furthermore, in some cases, combinations of the species exhibit synergy. Without committing to any particular theory, this suggests that various species of bacteria occupy different ecological niches and/or play differing roles in the regulation of tryptamine in the GI tract.

Applicants have also identified bacterial species associated with increasing 5-hydroxytryptamine levels (5HT). 5HT, or serotonin, is a brain neurotransmitter with broad impacts in host physiology due to its effects on gut motility and platelet aggregation, immune responses, cardiac function, and bone development. Approximately 95% of the body's 5HT is present in the gut, and the gut microbiome has been shown to modulate the host's 5HT levels (Yano et al., Cell 161:264-276, 2015; Ge et al., J. Transl. Med. 15:13, 2017). Dysregulation of 5HT has been implicated in a wide range of human diseases including irritable bowel syndrome (IBS), depression, anxiety, cardiovascular disease, and osteoporosis.

The invention provides compositions and methods for use in treating diseases and conditions that can benefit by the modulation of tryptophan and/or a tryptophan metabolite (e.g., tryptamine) in, e.g., the blood or GI tract of a mammal, such as a human. In some embodiments tryptamine levels are increased in the blood or gut of a mammal. In some embodiments, the tryptophan levels are decreased in the blood or GI tract of a mammal.

In some embodiments, the disclosure provides a bacterium comprising at least one gene or gene cassette encoding one or more enzymes for the production of tryptamine. In some embodiments, the bacterium comprises a gene sequence encoding a tryptophan decarboxylase (Trp decarboxylase).

In some embodiments, the disclosure provides compositions comprising one or more bacteria comprising at least one gene or gene cassette encoding one or more enzymes for the production of tryptamine (e.g., tryptophan decarboxylase). In some embodiments, the invention provides methods of using such compositions in order to alter (e.g., increase) tryptamine levels.

Tryptamine (TA) Compositions

Compositions useful in the inventions provided herein contain one or more (e.g., 2, 3, 4, 5, 6, 7, 8, 9, 10, 11, 12, or more) microorganisms that can modulate, e.g., increase, a tryptophan metabolite, e.g., tryptamine or 5-hydroxytryptamine, levels. In some cases, a useful microorganism can metabolize tryptophan to tryptamine or 5-hydroxytryptamine. In some cases, the microorganism can express a tryptophan decarboxylase or other enzyme, e.g., tyrosine carboxylase or phenylalanine carboxylase that can also metabolize tryptophan to tryptamine or 5-hydroxytryptamine. In some embodiments, one or more bacterial species are included in a composition and the combination can increase the amount of tryptamine or 5-hydroxytryptamine, e.g., in a culture or in a subject compared to the level when only one or the other species is provided in the culture or to the subject.

Non-limiting examples of microorganisms suitable for use in the inventions provided herein include those listed in Tables 1-5. The invention provides compositions including and methods of using any one of these microorganisms or combinations of, e.g., 2, 3, 4, 5, 6, 7, 8, 9, 10, 11, 12, or more of these microorganisms. In regard to combinations, the invention provides compositions comprising or consisting of any one or more of consortia 1 to 47, as indicated in Table 6 (compositions 1-47). The invention further includes methods of using these compositions, as described herein.

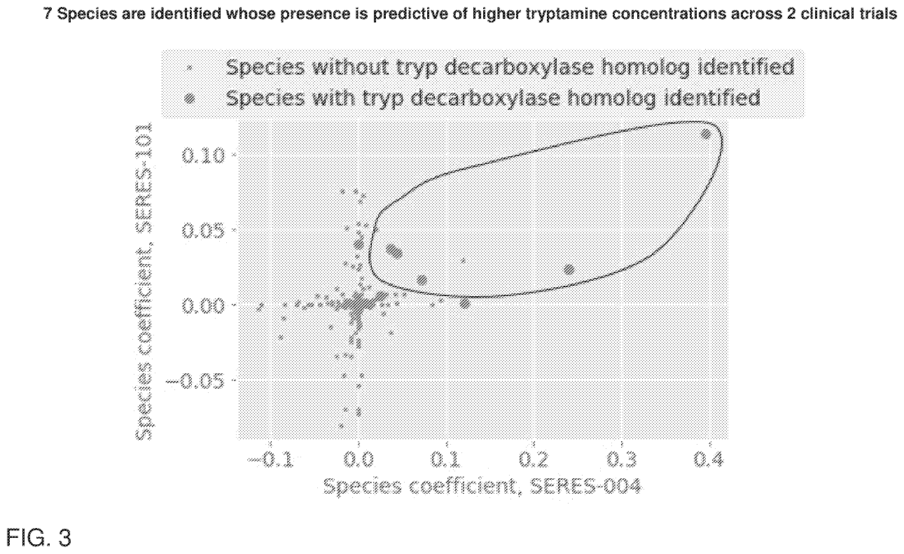

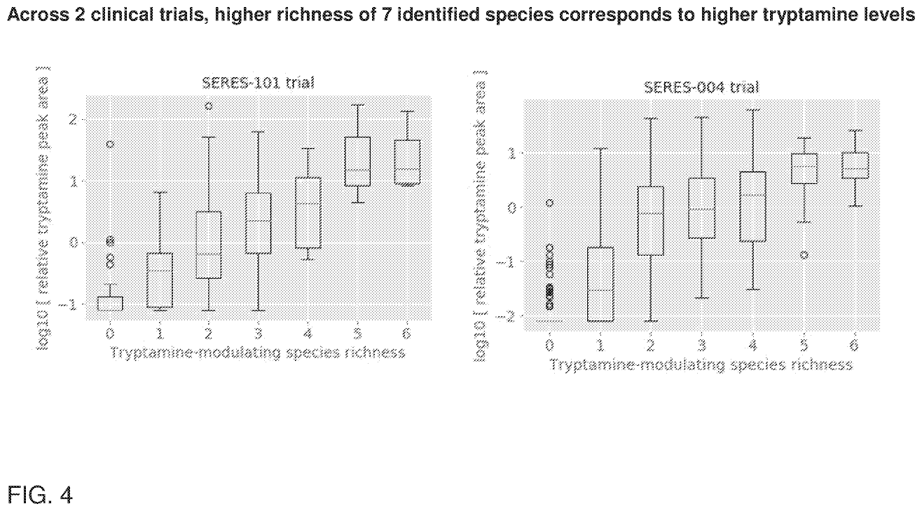

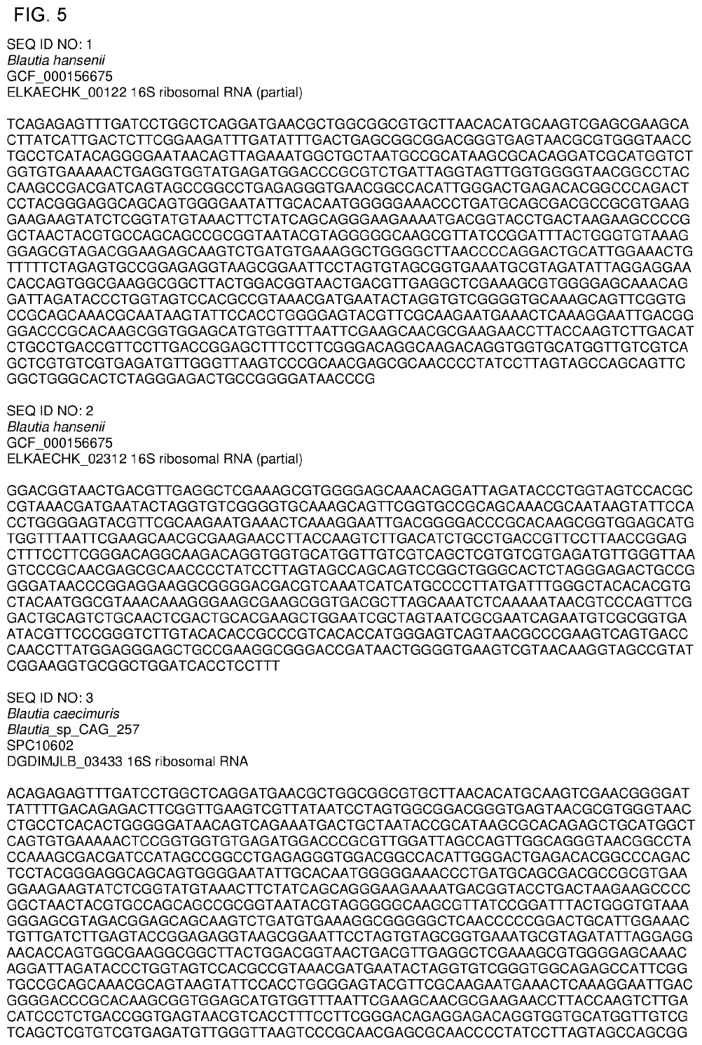

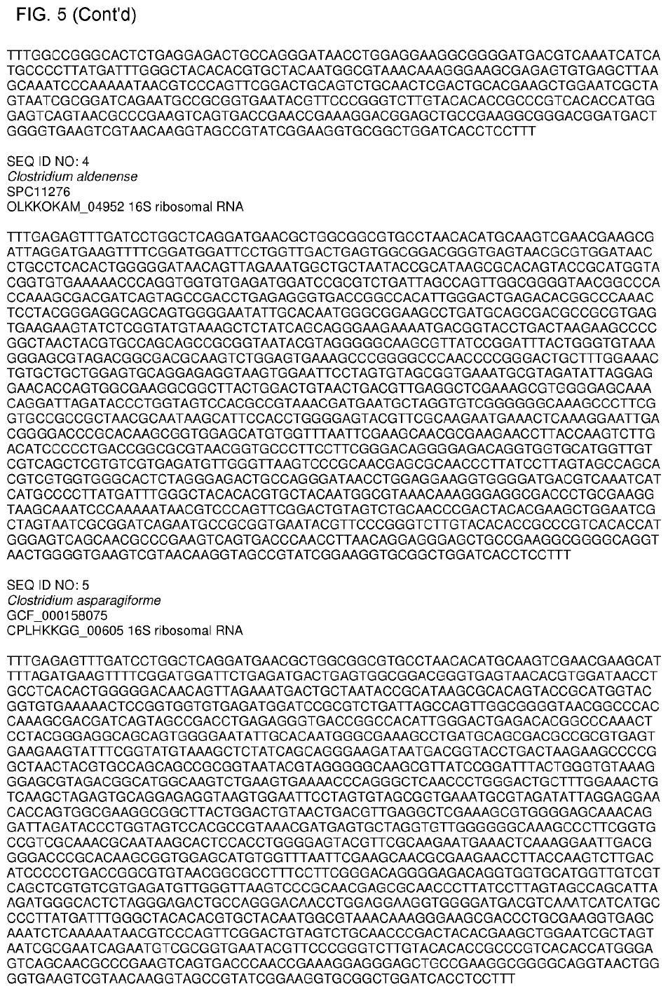

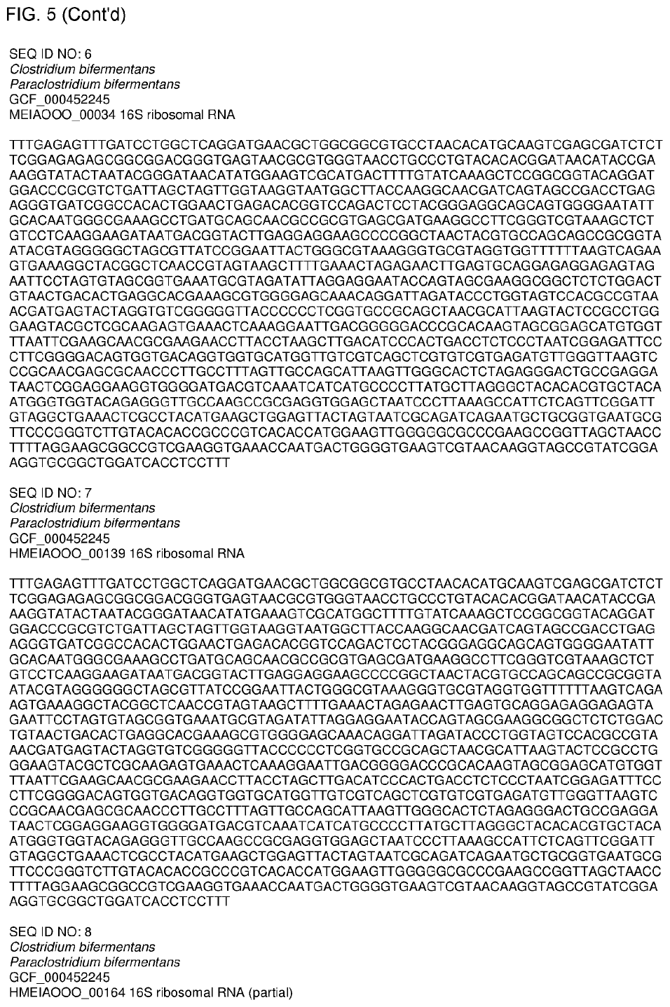

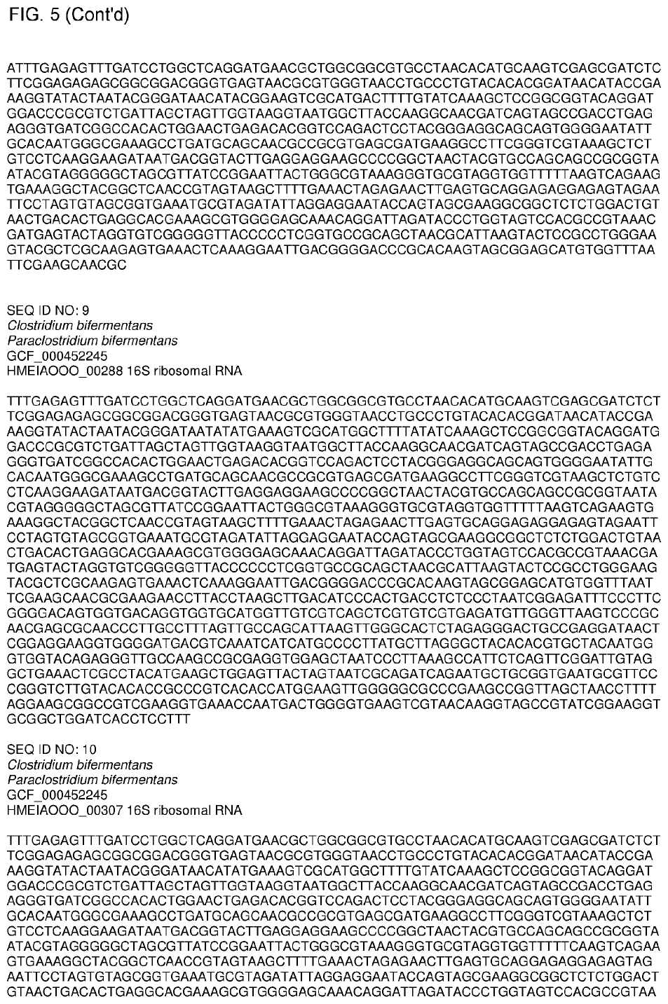

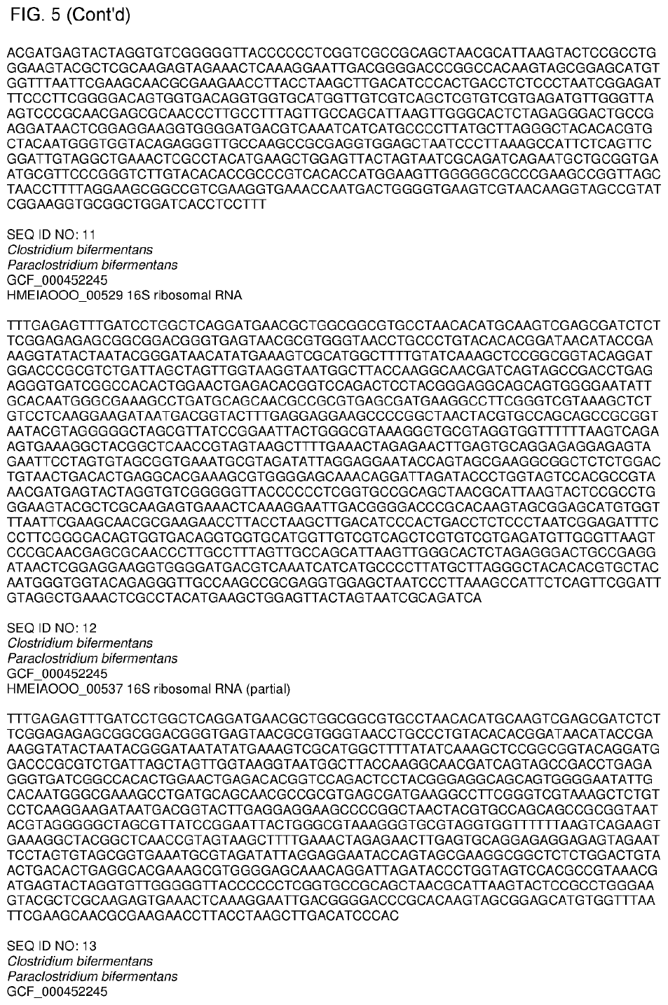

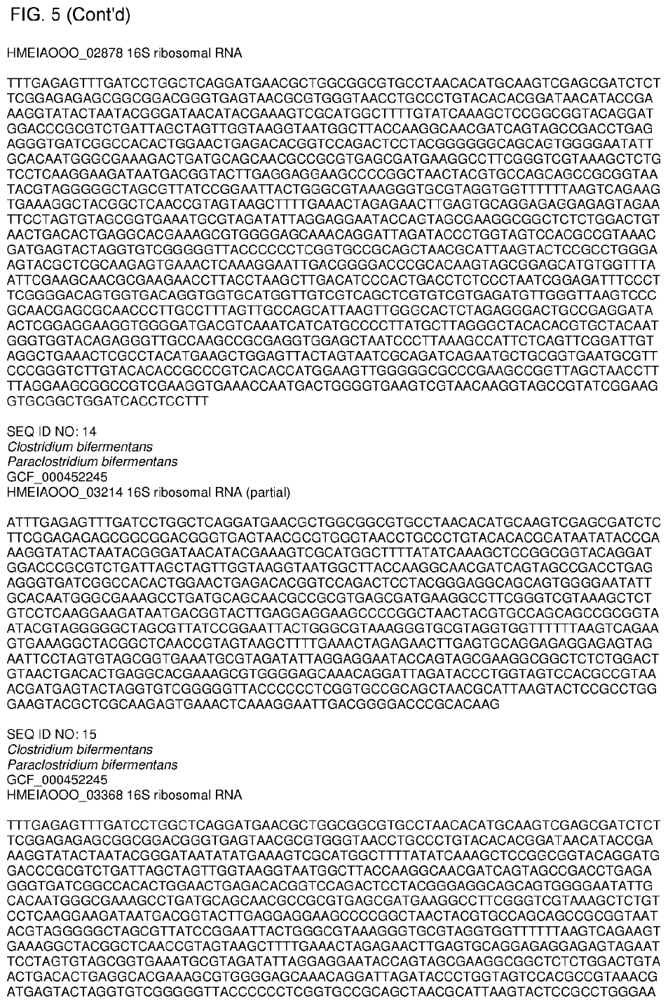

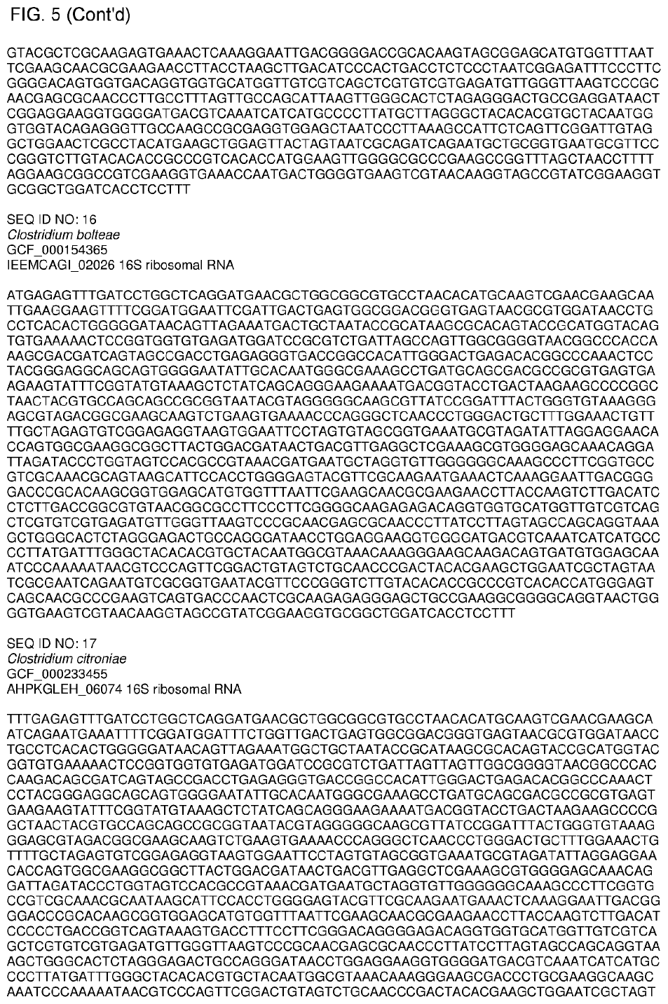

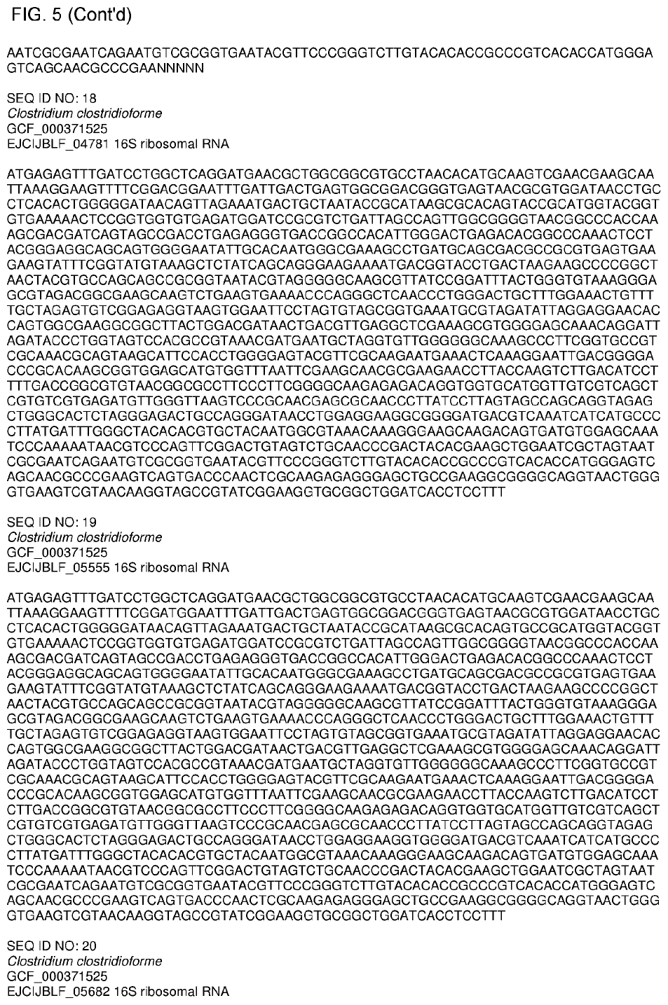

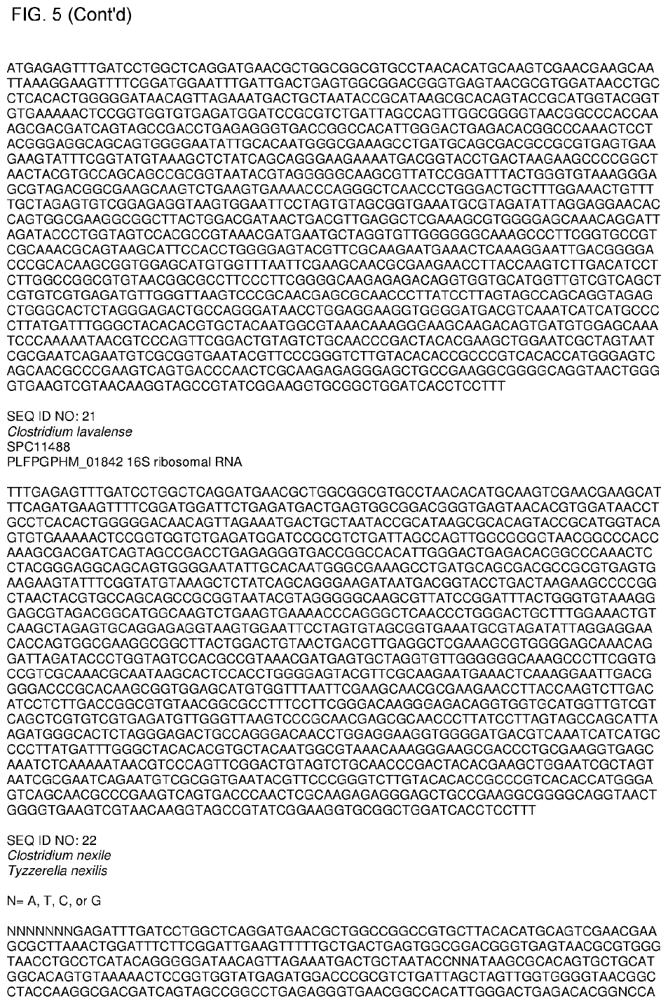

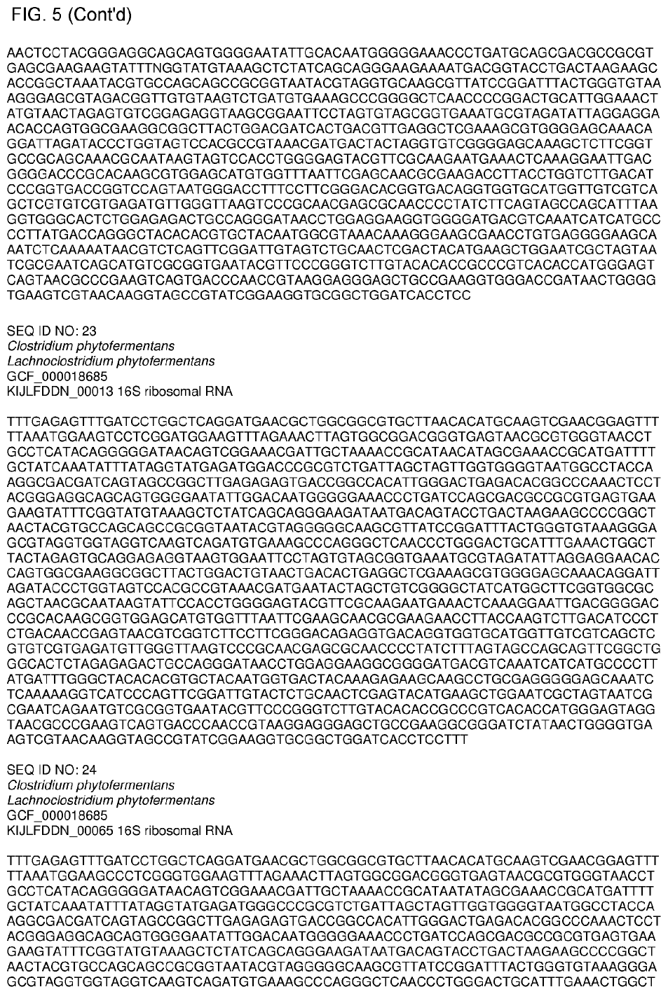

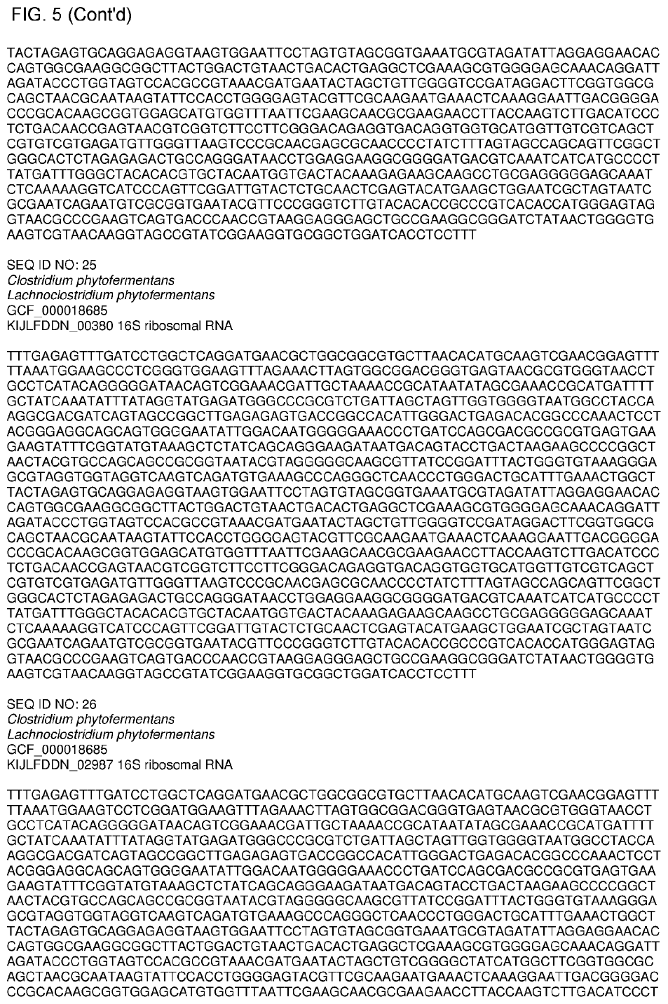

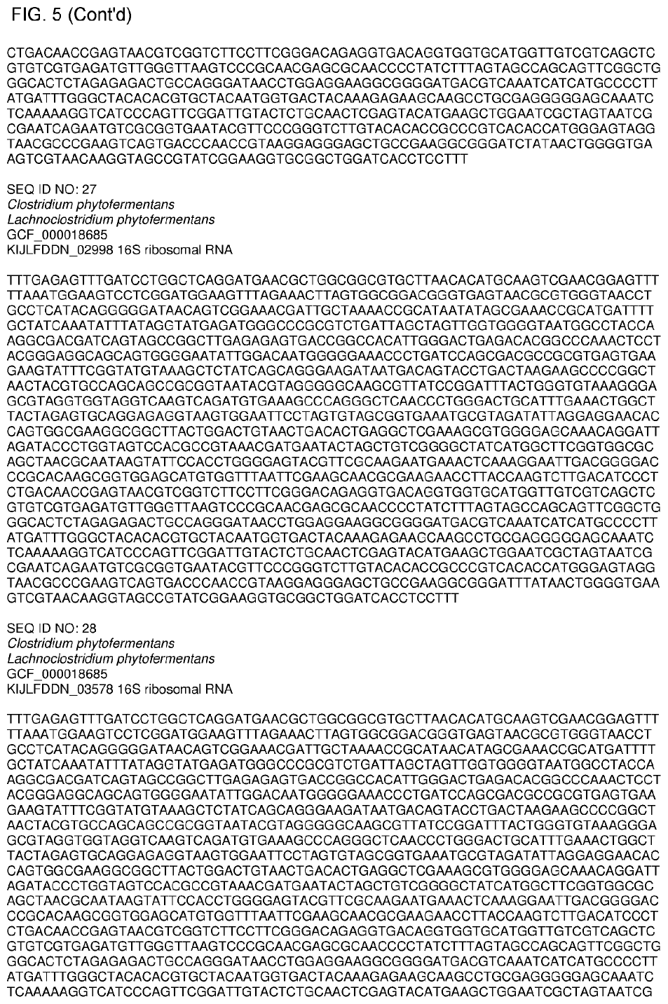

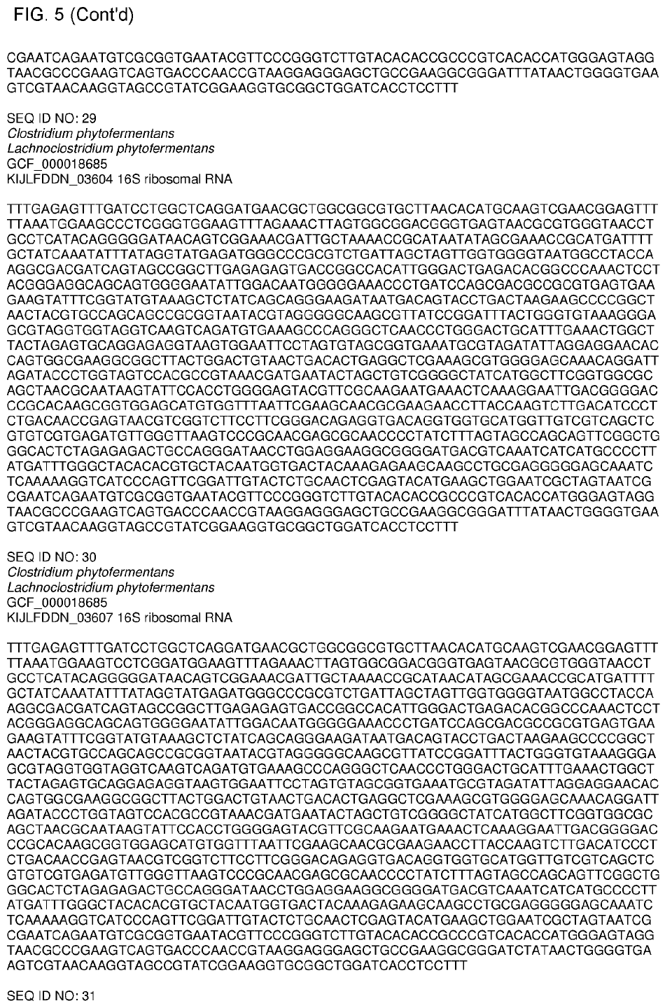

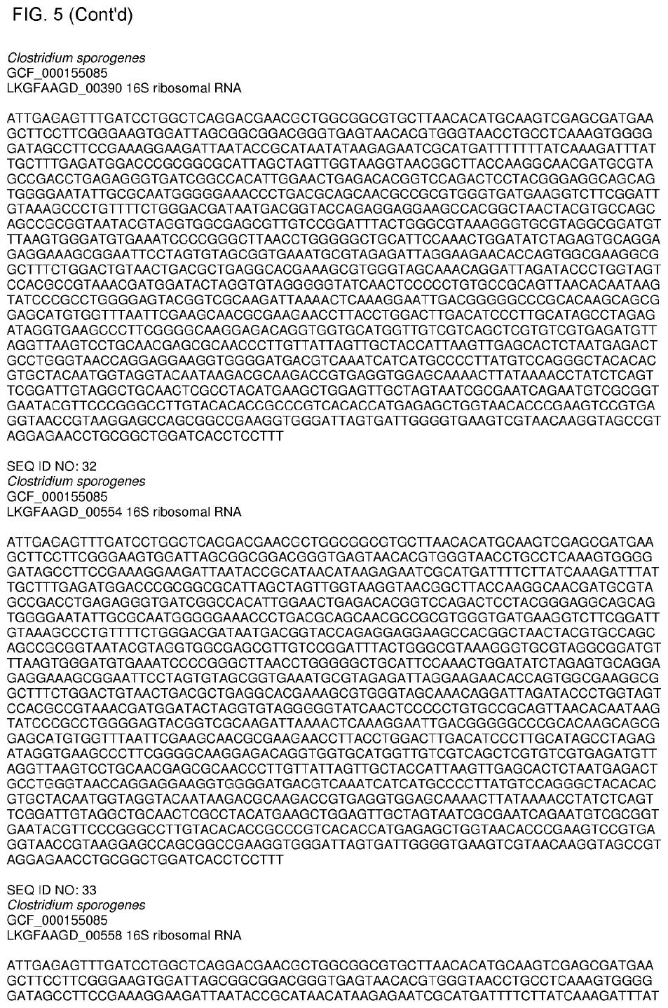

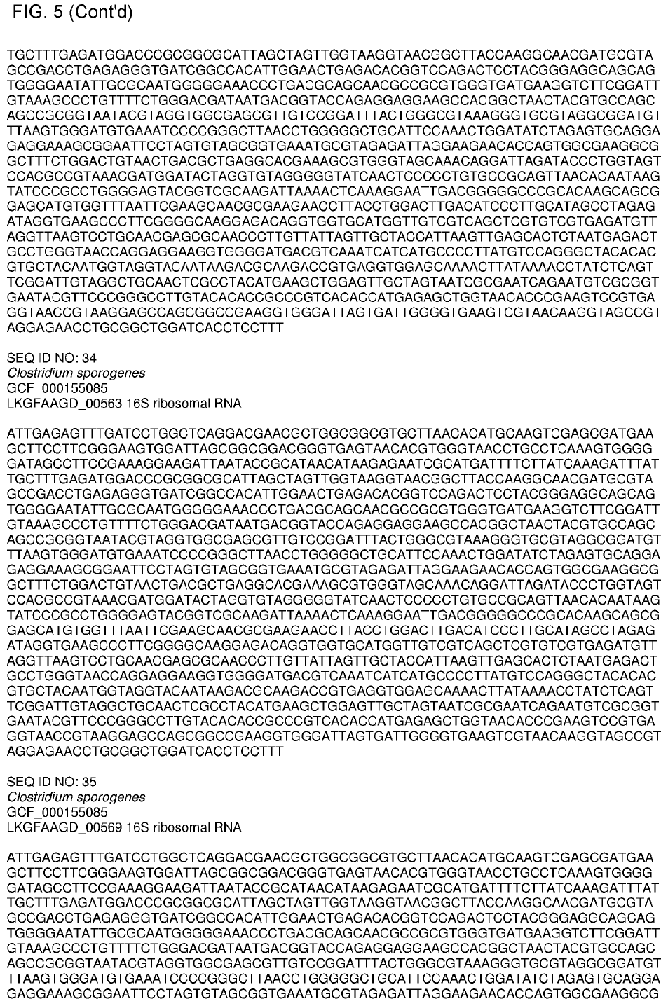

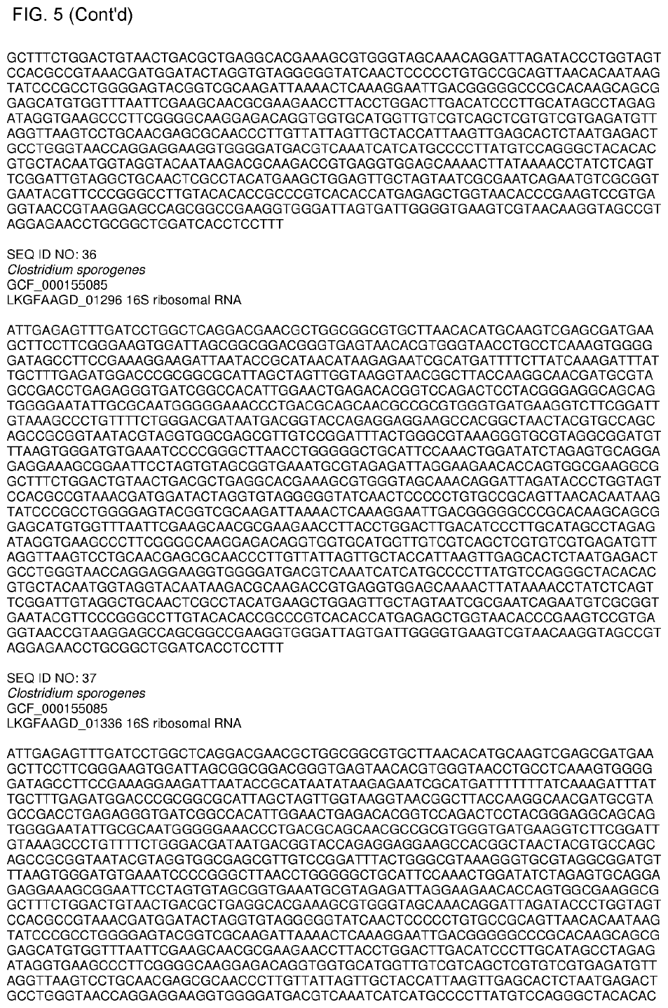

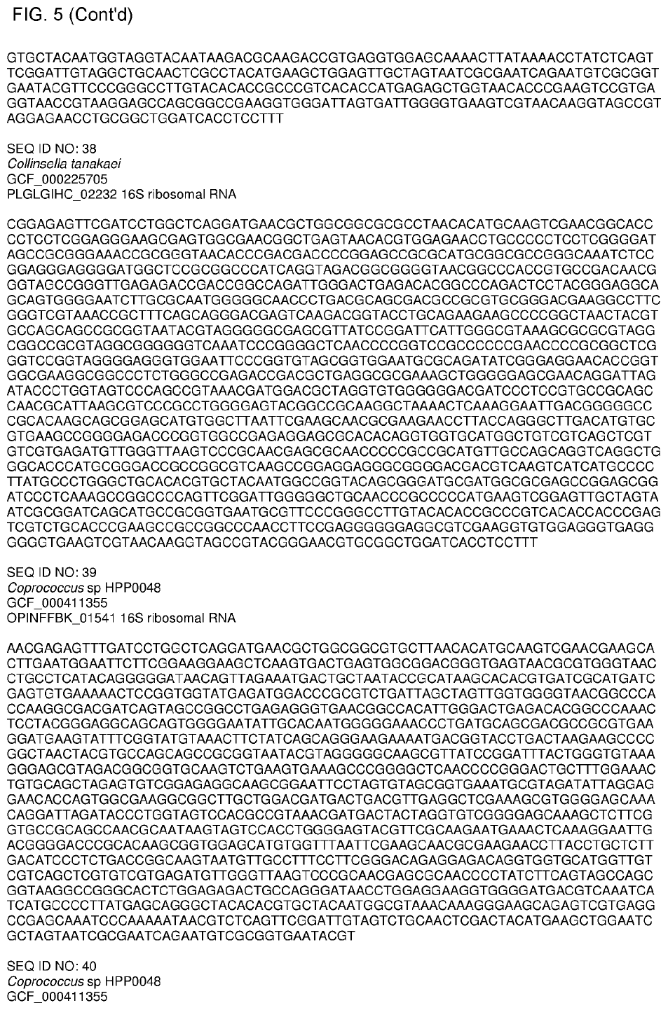

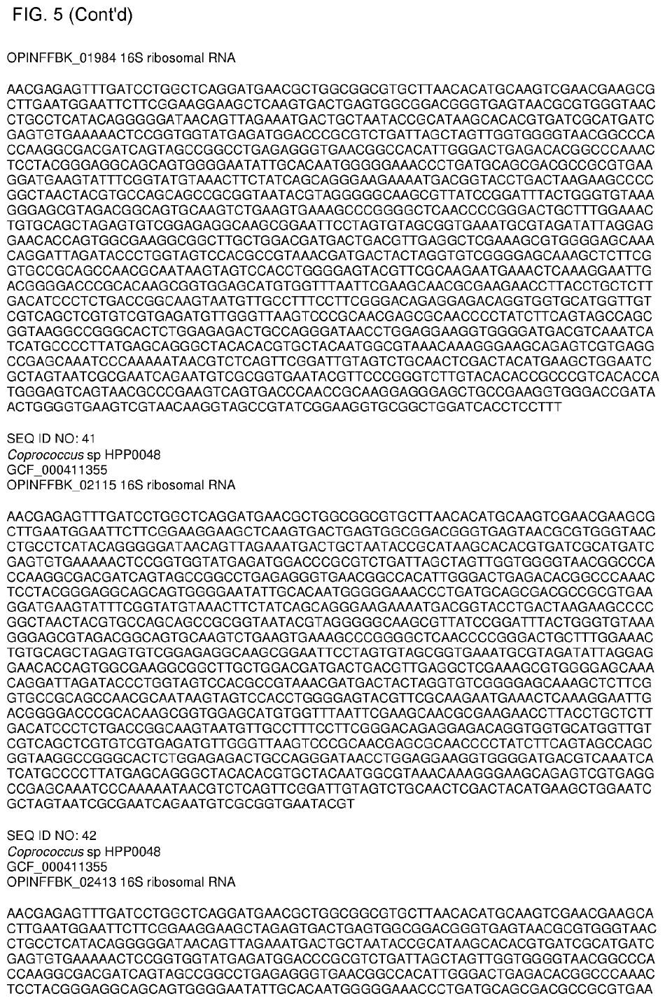

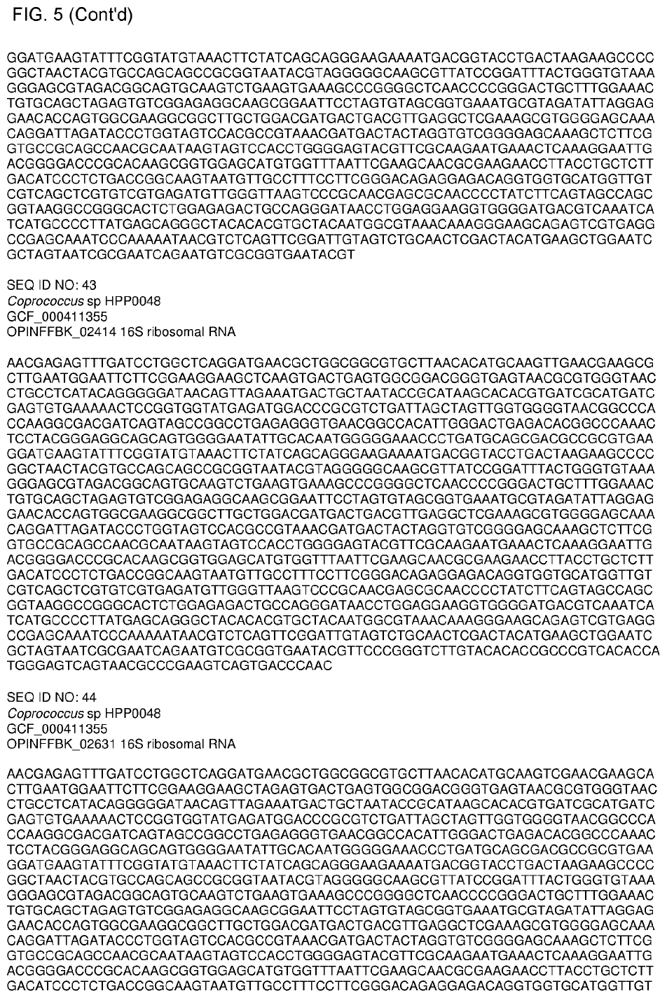

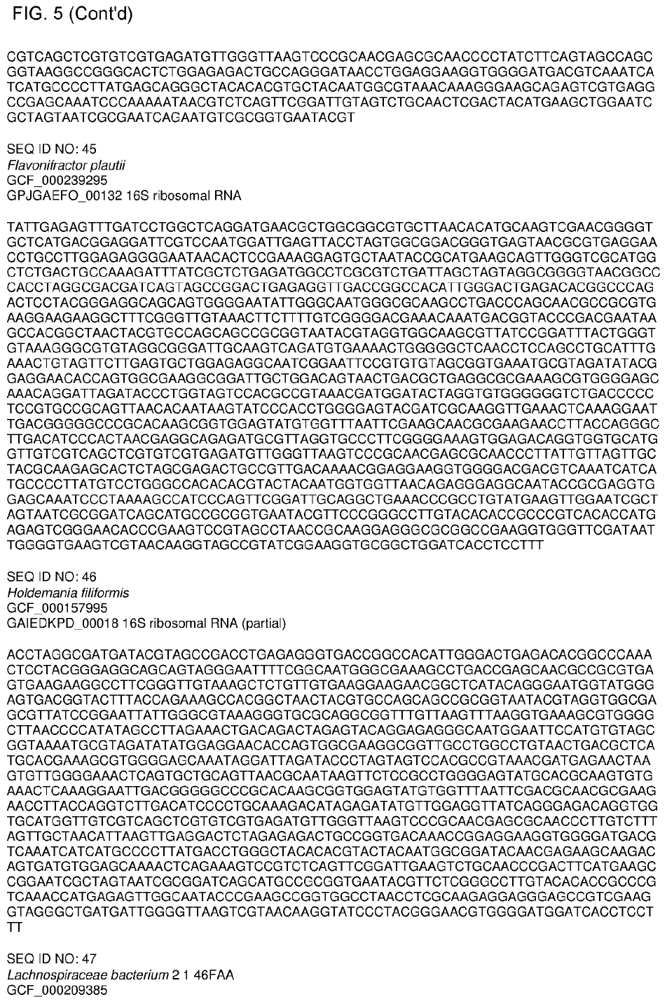

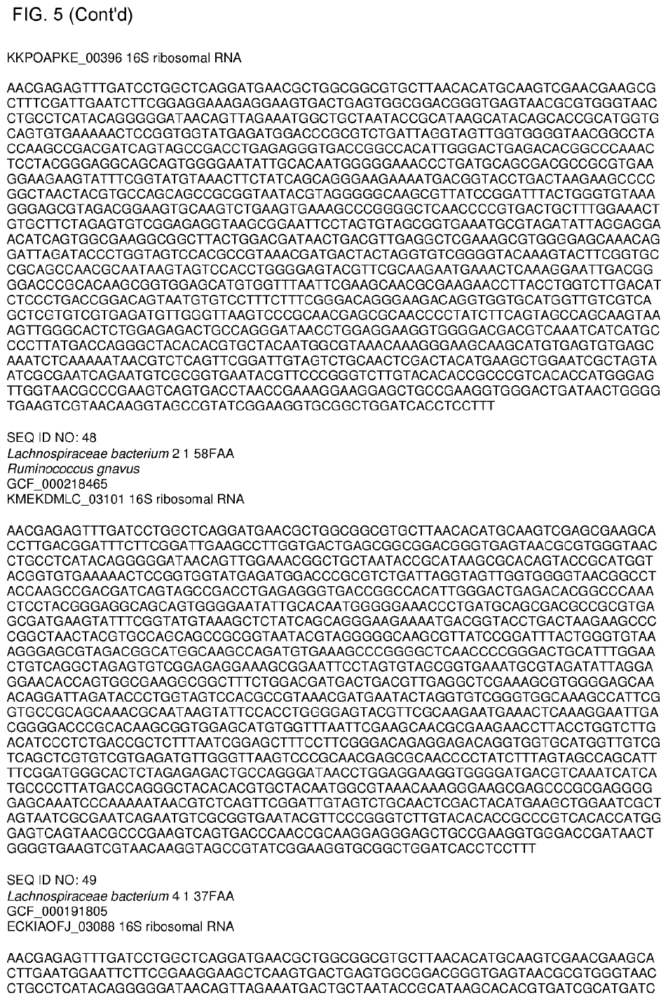

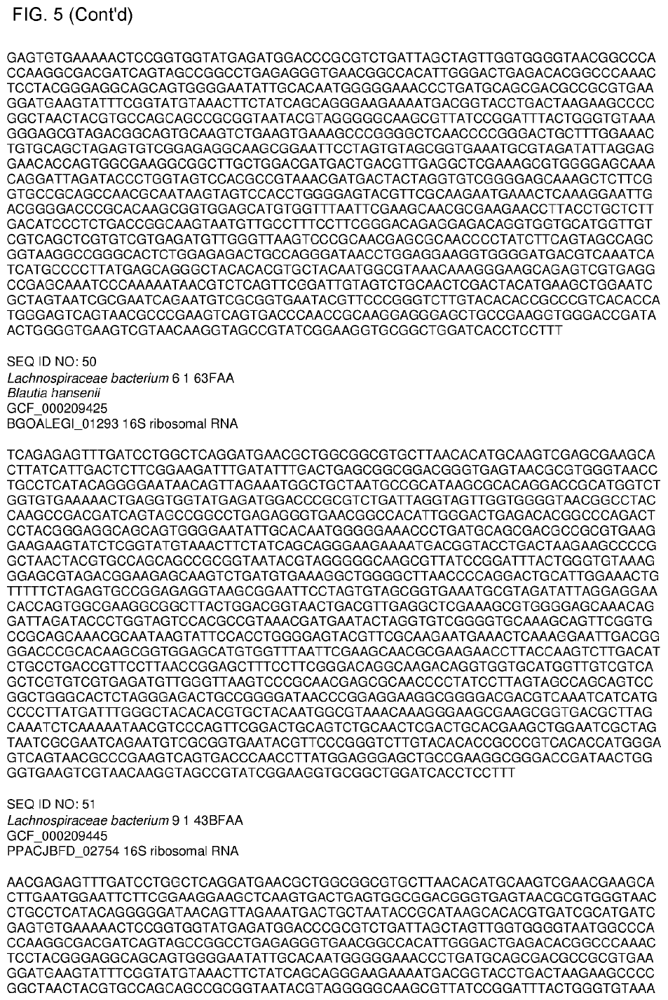

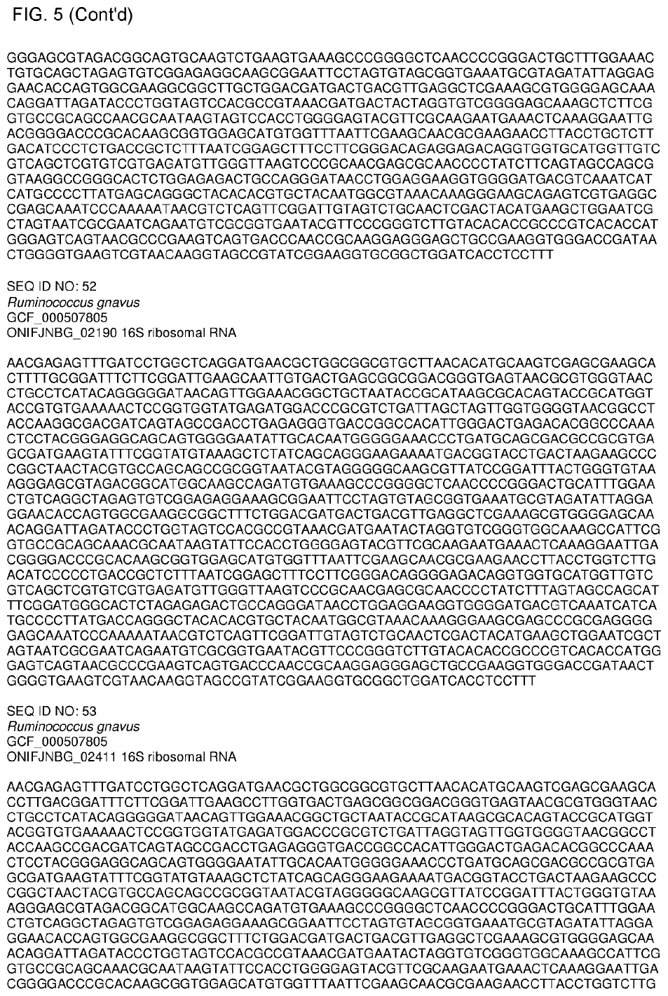

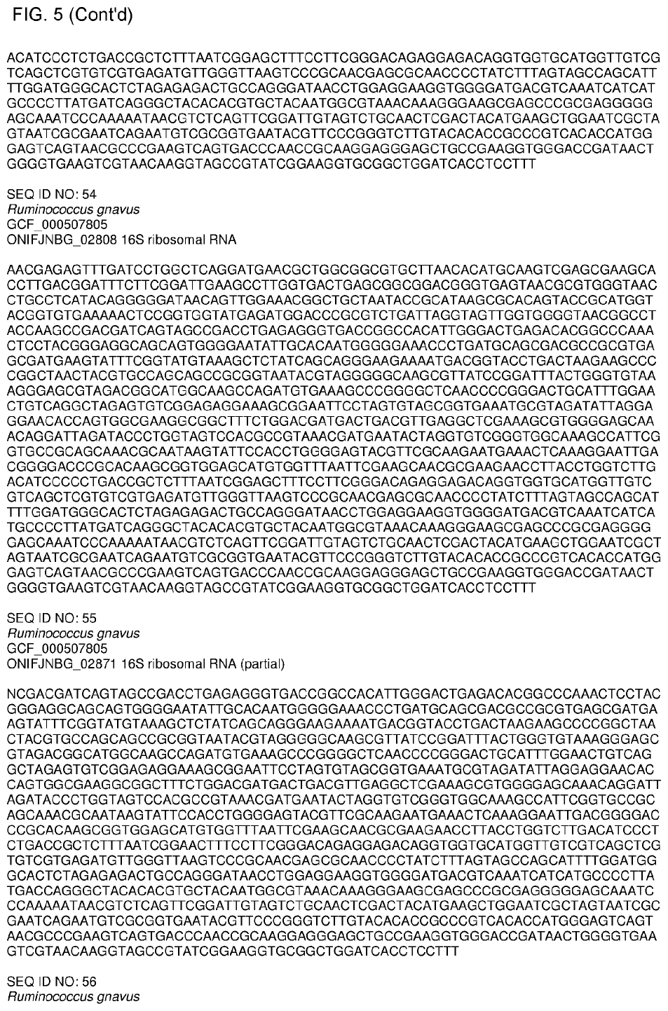

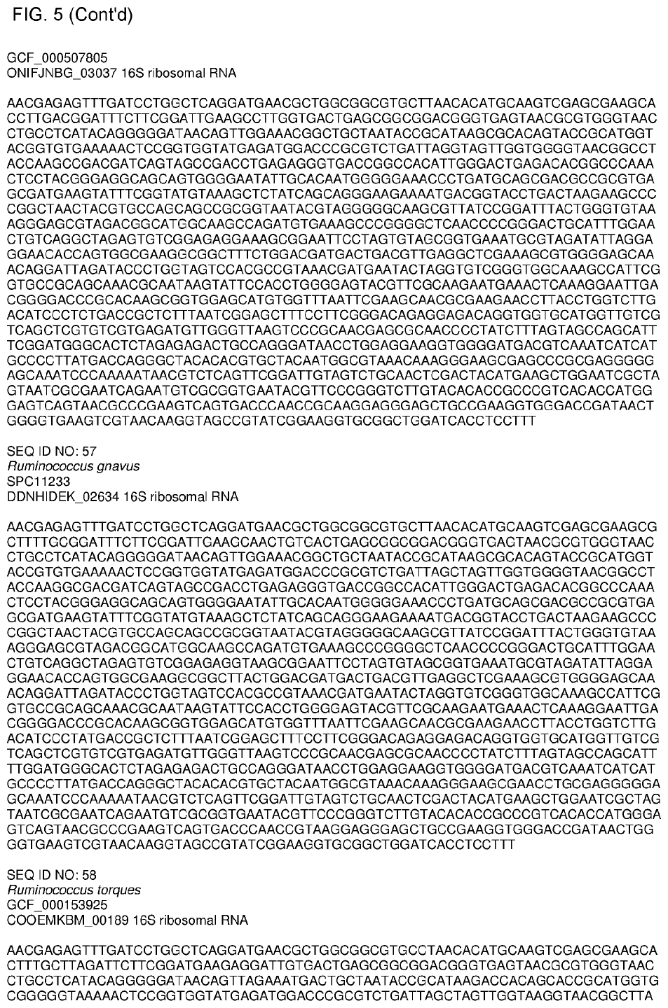

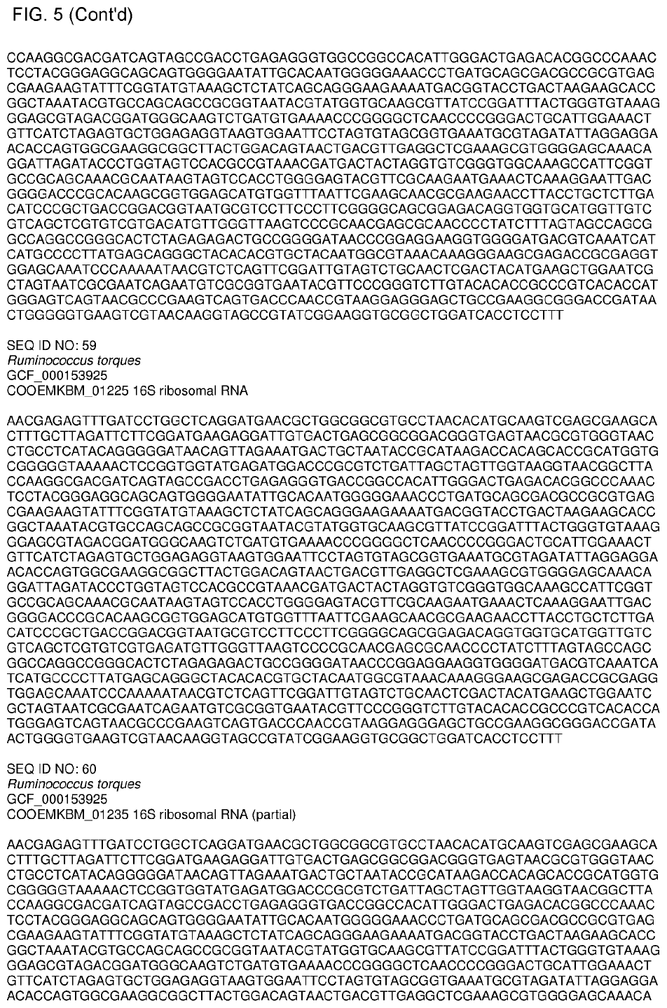

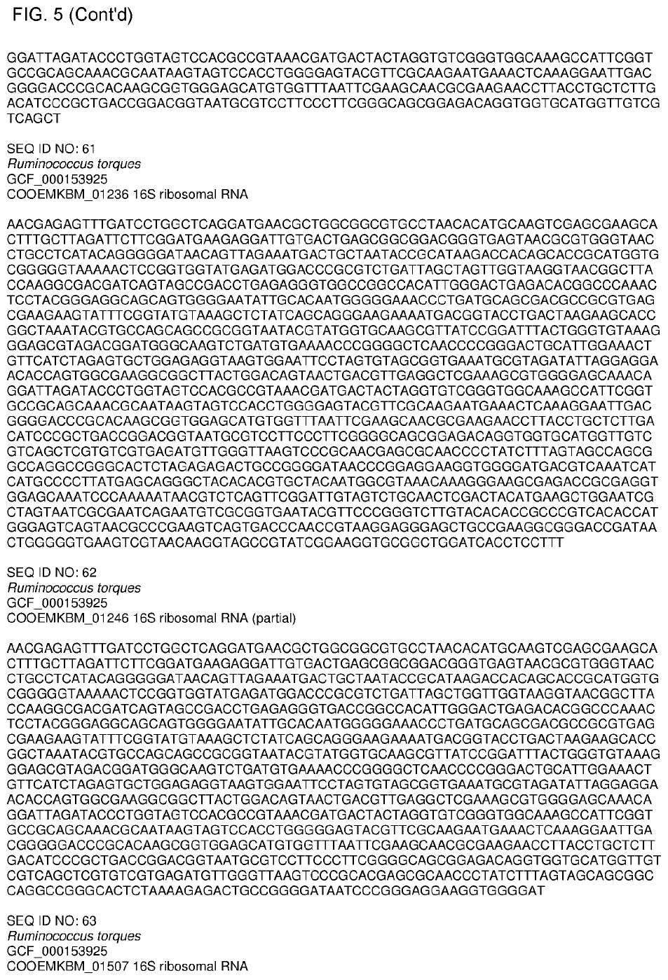

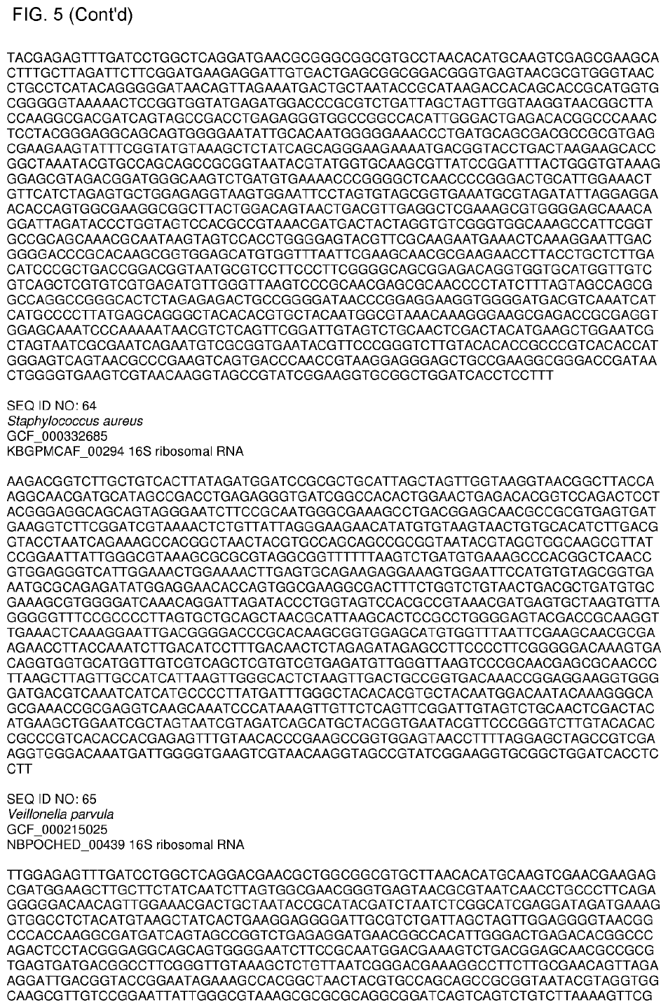

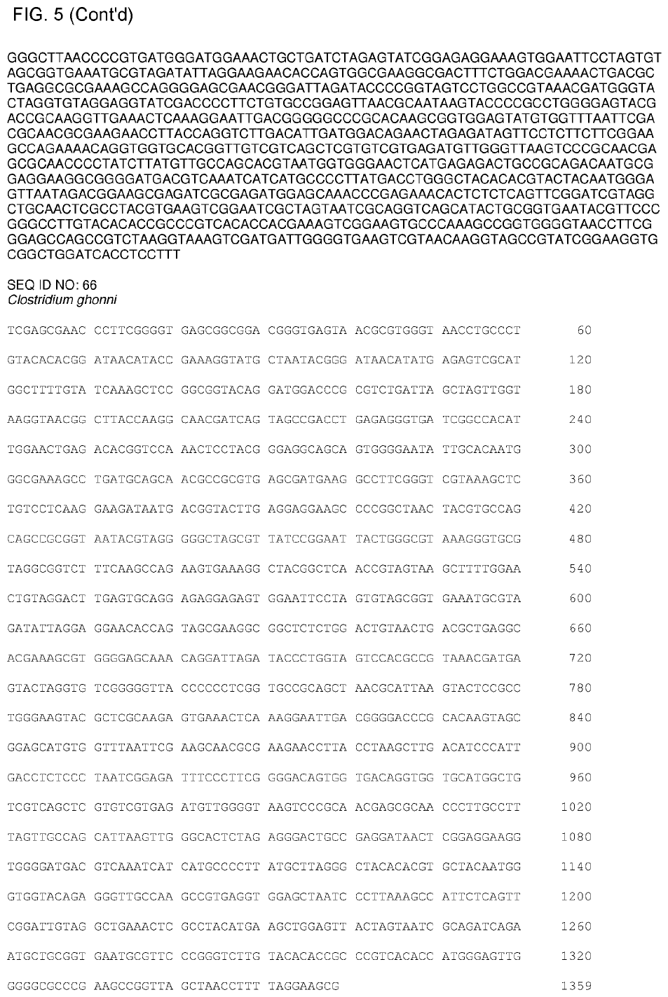

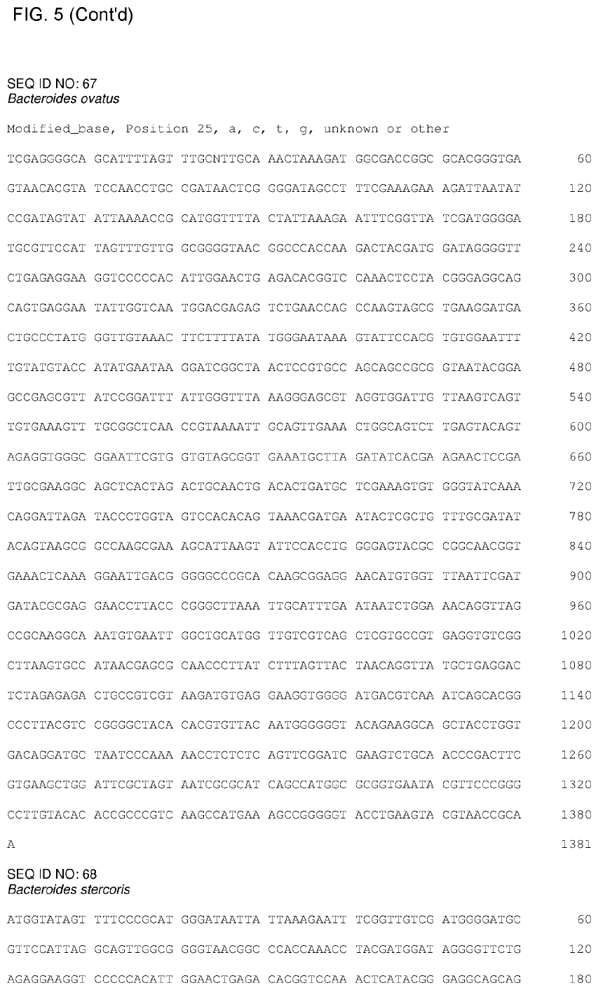

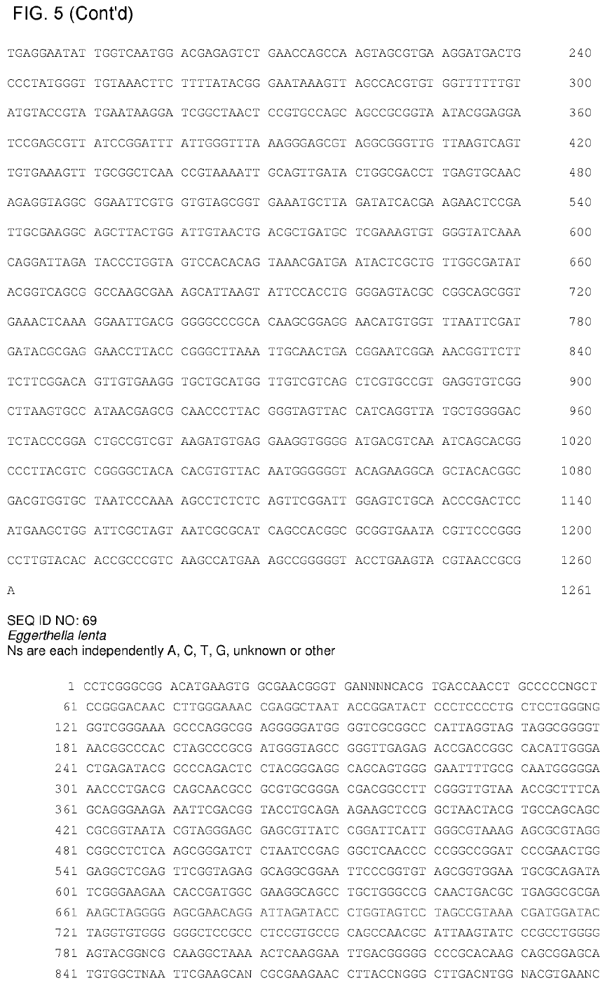

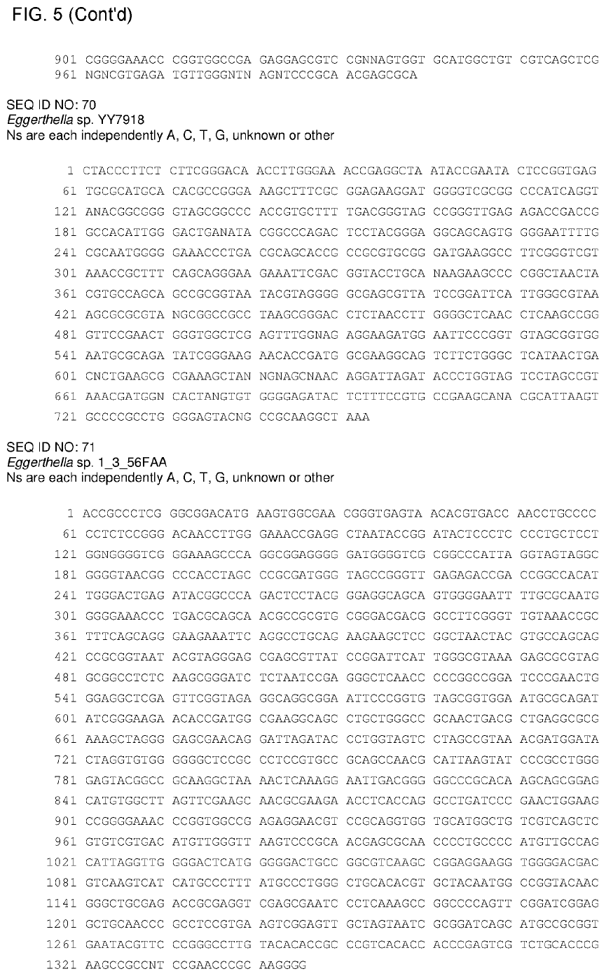

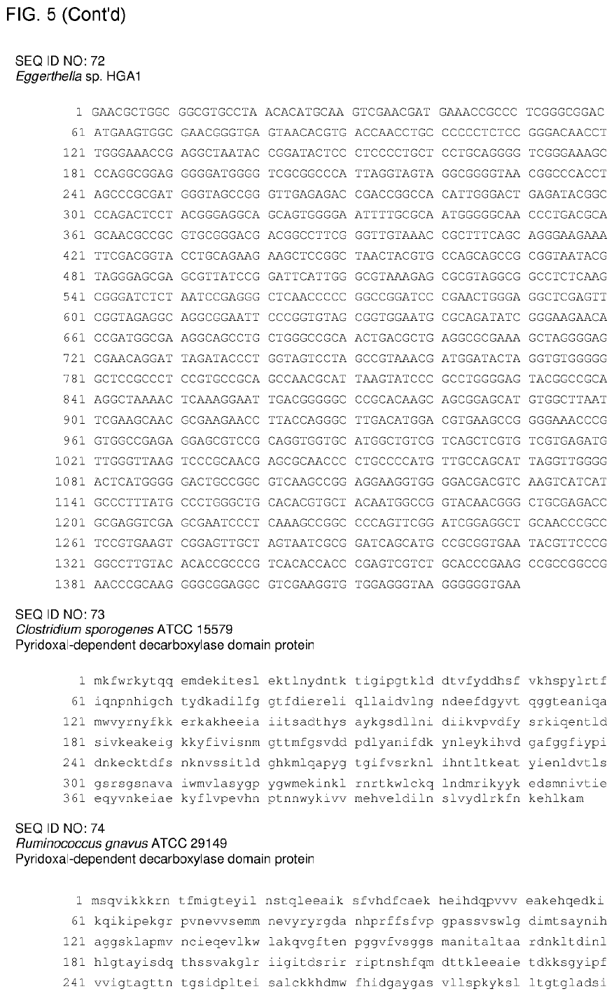

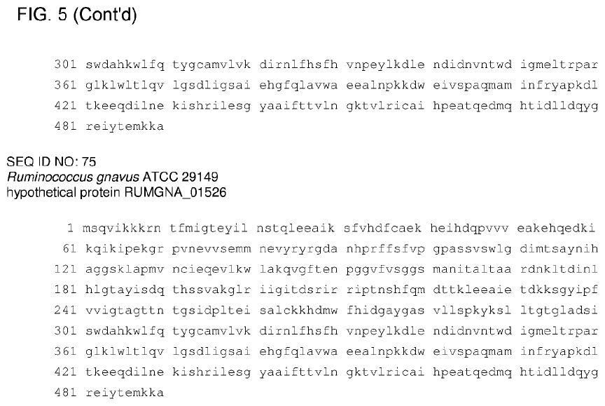

Table 1 provides non-limiting examples of species that can be included in the compositions of the invention, as well as references to 16S rDNA sequences that can be used for identifying these species and additional examples of related species and strains. The species listed in Table 1 were found to be predictive of tryptamine in two clinical trials (see, e.g., Example 2, FIG. 3, and Table 9). Compositions of the invention can include 1, 2, 3, 4, 5, 6, or 7 of the species listed in Table 1, in any combination with each other or other species listed herein (e.g., in different tables). 16S rDNA sequence information for these and other species useful in the compositions and methods of the invention are provided in FIG. 5.

TABLE 1

| | | Exemplary | |
|---|---|---|---|
| | Exemplary | GenBank 16S | 16S rDNA |
| Species | OTU** | rDNA | SEQ ID NO |
| Ruminococcus_gnavus | ATCC 29149; | NR_118690.1 | 52-57 |
| | ATCC 35913 | | |
| Lachnospiraceae_bacterium_9_1_43BFAA | | LV535612.1 | 51 |
| Eggerthella_unclassified | DSM 2242; | | 69-72 |
| | ATCC 25559D-5 | | |
| Clostridium_{—}nexile | ATCC 27757 | | 22 |
| Lachnospiraceae_bacterium_6_1_63FAA | ATCC 27752 | | 50 |
| (Blautia hansenii) | | | |
| Ruminococcus_torques | ATCC 27756; | NR_036777.1; | 58-63 |
| | ATCC 35915 | AB910746.1 | |

**Exemplary OTUs in the Tables herein have 16S rDNA sequences that are at least 97% identical to the listed strains. In addition to the exemplary deposited microorganisms, microorganisms can be obtained from the sources of type strains as indicated in publications thereof.

Table 2 provides additional, non-limiting examples of species that can be used in the compositions and methods of the invention, as well as references to 16S rDNA sequences that can be used for identifying these species and additional examples of related species and strains identified by in vitro screening. Compositions of the invention can include 1, 2, 3, 4, 5, 6, 7, 8, 9, 10, or 11 of the species listed in Table 2, in any combination with each other or other species listed herein (e.g., in different tables). 16S rDNA sequence information for these and other species useful in the compositions and methods of the invention are provided in FIG. 5. These sequences can be used to identify these and related species and strains.

TABLE 2

| | Exemplary 16S | |
|---|---|---|
| | rDNA sequence | |
| | identifier(s) | Exemplary |
| Species | (SEQ ID NO) | OTU |
| Clostridium ghonii | 66 | ATCC 25757 |
| Flavonifractor plautii | 45 | ATCC 29863 |
| Ruminococcus gnavus | 52-57 | ATCC 29149; |
| | | ATCC 39143 |
| Bacteroides ovatus | 67 | ATCC 8483; |
| | | ATCC700292 |
| Bacteroides stercoris | 68 | ATCC 43183 |
| Clostridium sporogenes | 31-37 | ATCC 11437D-5; |
| | | ATCC 3584; |
| | | ATCC 7955 |

Table 3 provides additional, non-limiting examples of species that can be used in the compositions and methods of the invention, as well as references to 16S rDNA sequences that can be used for identifying these species and additional examples of related species and strains. Compositions of the invention can include 1, 2, 3, or 4 of the species listed in Table 3, in any combination with each other or other species listed herein (e.g., in different tables). 16S rDNA sequence information for these and other species useful in the compositions and methods of the invention are provided in FIG. 5. These sequences can be used to identify these and related species and strains.

TABLE 3

| | Exemplary 16S | |
|---|---|---|
| | rDNA sequence | |
| | identifier(s) | Exemplary |
| Species | (SEQ ID NO) | OTU |
| Lachnospiraceae_bacterium_2_1_58FAA | 48 | ATCC 29149; |
| (Ruminococcus gnavus) | | ATCC 39143 |
| Clostridium_aldenense | 4 | ATCC BAA-1318 |
| Clostridium_citroniae | 17 | ATCC BAA-1317; |
| | | DSM 19261 |
| Clostridium_clostridioforme | 18-20 | ATCC PTS-25537; |
| | | ATCC 29084; |
| | | ATCC 25537 |

Table 4 provides additional, non-limiting examples of species that can be used in the compositions and methods of the invention, as well as references to 16S rDNA sequences that can be used for identifying these species and additional examples of related species and strains. Compositions of the invention can include 1, 2, 3, or 4 of the species listed in Table 4, in any combination with each other or other species listed herein (e.g., in different tables). 16S rDNA sequence information for these and other species useful in the compositions and methods of the invention are provided in FIG. 5. These sequences can be used to identify these and related species and strains.

TABLE 4

| | Exemplary 16S | |
|---|---|---|
| | rDNA sequence | |
| | identifier(s) | Exemplary |
| Species | (SEQ ID NO) | OTU |
| Flavonifractor_plautii | 45 | ATCC 29863 |
| Veillonella_parvula | 65 | ATCC 10790D-5; |
| | | ATCC 35184; |
| | | ATCC 10790; |
| | | ATCC 17745 |
| Blautia_sp_CAG_257 | 3 | See GenBank: |
| | | CDA06774.1 |
| | | for source |
| Clostridium_bolteae | 16 | ATCC BAA-613 |

Table 5 provides additional, non-limiting examples of species that can be used in the compositions and methods of the invention, as well as references to 16S rDNA sequences that can be used for identifying these species and additional examples of related species and strains. Compositions of the invention can include 1, 2, 3, 4, 5, 6, 7, 8, 9, 10, 11, or 12 of the species listed in Table 5, in any combination with each other or other species listed herein (e.g., in different tables). 16S rDNA sequence information for these and other species useful in the compositions and methods of the invention are provided in FIG. 5. These sequences can be used to identify these and related species and strains.

TABLE 5

| | Exemplary 16S | |
|---|---|---|
| | rDNA sequence | |
| | identifier(s) | Exemplary |
| Species | (SEQ ID NO) | OTUs |
| Blautia_{—}hansenii | 1, 2 | ATCC 27752 |
| Lachnospiraceae_bacterium_2_1_46FAA | 47 | |
| Coprococcus_sp_HPP0048 | 39-44 | Riken deposit numbers: |
| | | JCM 1429, JCM 6483, |
| | | JCM 6515, JCM, 30896, |
| | | JCM 31500, JCM 31501 |
| Collinsella_tanakaei | 38 | DSM 22478 |
| Clostridium_sporogenes | 31-37 | ATCC 14437D-5; |
| | | ATCC 3584 |
| Clostridium_phytofermentans | 23-30 | ATCC 700394; ATCC |
| (Lachnoclostridium phytofermentans) | | 700394D-5 |
| Clostridium_bifermentans | 6-15 | ATCC 17837; ATCC |
| (Paraclostridium bifermentans) | | 19299; ATCC 17839; |
| | | ATCC 17840 |
| Staphylococcus_aureus | 64 | ATCC 25923D-5 |
| (i.e., non-methicillin resistant S. aureus) | | |
| Lachnospiraceae_bacterium_4_1_37FAA | 49 | Riken deposit numbers: |
| | | JCM 1429, JCM 6483, |
| | | JCM 6515, JCM, 30896, |
| | | JCM 31500, JCM 31501 |
| Clostridium_asparagiforme | 5 | DSM 15981 |
| Clostridium_lavalense | 21 | DSM 19851 |
| Holdemania_filiformis | 46 | ATCC 51649 |

As noted above, the invention includes compositions including combinations of bacterial species, as described herein, methods of using these compositions for altering (e.g., increasing) tryptamine levels, and methods of treating diseases and conditions in which altering (e.g., increasing) tryptamine levels would be advantageous. Specific, non-limiting examples of 47 of such combination compositions are set forth in Table 6.

TABLE 6

| | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| Ruminococcus_{—}torques | x | | | | | x | | x | x | x | X | x | x | x |
| Lachnospiraceae_bacterium_6_1_63FAA | x | | | x | | | | | | | | | x | x |
| (Blautia hansenii) | | | | | | | | | | | | | | |
| Lachnospiraceae_bacterium_9_1_43BFAA | | x | | | | | | | x | | | | | |
| Ruminococcus_{—}gnavus | | x | x | x | x | | | | | x | X | | | |
| (strain 1) | | | | | | | | | | | | | | |
| Clostridium_{—}nexile | | | x | | | | x | x | | | | x | | x |
| Eggerthella_unclassified | | | | | x | x | x | | | | x | x | x | |
| Ruminococcus gnavus | | | | | | | | | | | | | | |
| (strain 2) | | | | | | | | | | | | | | |

| | 15 | 16 | 17 | 18 | 19 | 20 | 21 | 22 | 23 | 24 |
|---|---|---|---|---|---|---|---|---|---|---|
| Ruminococcus_{—}torques | | | x | | | x | x | x | | x |
| Lachnospiraceae_bacterium_6_1_63FAA | x | | | x | | | x | | | x |
| (Blautia hansenii) | | | | | | | | | | |
| Lachnospiraceae_bacterium_9_1_43BFAA | | x | | x | | | | x | x | |
| Ruminococcus_{—}gnavus | x | x | x | x | x | x | x | x | x | x |
| (strain 1) | | | | | | | | | | |
| Clostridium_{—}nexile | | x | x | | x | | | | x | |
| Eggerthella_unclassified | x | | | | x | | | | x | x |
| Ruminococcus gnavus | | | | | | x | | | | |
| (strain 2) | | | | | | | | | | |

| | 25 | 26 | 27 | 28 | 29 | 30 | 31 | 32 | 33 | 34 | 35 | 36 | 37 | 38 |
|---|---|---|---|---|---|---|---|---|---|---|---|---|---|---|
| Ruminococcus_{—}torques | x | x | x | x | x | x | x | x | x | x | x | x | x | x |
| Lachnospiraceae_bacterium_6_1_63FAA | | | x | | x | x | | | x | | | x | | x |
| (Blautia hansenii) | | | | | | | | | | | | | | |
| Lachnospiraceae_bacterium_9_1_43BFAA | | x | | x | | | x | | x | | x | | | x |
| Ruminococcus_{—}gnavus | x | x | | x | x | x | x | x | x | x | x | x | x | x |
| (strain 1) | | | | | | | | | | | | | | |
| Clostridium_{—}nexile | x | | x | | | x | x | | | x | x | x | x | x |
| Eggerthella_unclassified | x | x | x | | | | | x | | | x | x | x | |
| Ruminococcus gnavus | | | | x | x | | | x | | x | | | x | |
| (strain 2) | | | | | | | | | | | | | | |

| | 39 | 40 | 41 | 42 | 43 | 44 | 45 | 46 | 47 |
|---|---|---|---|---|---|---|---|---|---|
| Ruminococcus_{—}torques | x | x | x | x | x | x | x | x | x |
| Lachnospiraceae_bacterium_6_1_63FAA | | x | x | | x | x | x | | x |
| (Blautia hansenii) | | | | | | | | | |
| Lachnospiraceae_bacterium_9_1_43BFAA | x | | x | x | x | | x | x | x |
| Ruminococcus_{—}gnavus | x | x | x | x | x | x | x | x | x |
| (strain 1) | | | | | | | | | |
| Clostridium_{—}nexile | | | | x | | x | x | x | x |
| Eggerthella_unclassified | x | x | | | x | x | x | x | |
| Ruminococcus gnavus | x | x | x | x | x | x | | x | x |
| (strain 2) | | | | | | | | | |

Additional microorganisms that can be useful in the inventions are Clostridium argentinese, Clostridium polymyxa, Erysipelothrix rhusiopathiae, Staphylococcus epidermidis, Staphylococcus haemolyticus, Staphylococcus carnosus, Corynebacterium pyruviciproducens, Staphylococcus pseudintermedius, Corynebacterium glucuronolyticum, Bacillus atrophaeus, Bacillus weihenstephanensis, and non-pathogenic Bacillus anthracis. Optionally, one or more of these additional microorganisms can be used in combination with any of the combinations described in the immediately preceding paragraph.

In some embodiments, a useful microorganism comprises a DNA sequence predicted to encode a protein having at least 93% identity to a reference Trp decarboxylase sequence (full length or the tryptophan binding region). In some embodiments, the microorganism comprises a DNA sequence predicted to encode a protein having at least, 40%, 50%, 55%, 60%, 65%, 70%, 75%, 80%, 85%, 90%, 92%, 93%, 94%, 95%, 98%, 99%, or 100% identity to a reference Trp decarboxylase sequence. Non-limiting examples of reference sequences are GenBank: EDU35915.1 (SEQ ID NO: 73), GenBank: EDN78222.1 (SEQ ID NO: 74), GenBank ZP_02040762 (SEQ ID NO: 75).

In some embodiments, a useful microorganism comprises a 16S rDNA sequence sharing sequence identity to one or more 16S rDNA sequences of a species listed herein (e.g., one or more of the species listed in one or more of Tables 1-6; see, e.g., FIG. 5 and SEQ ID NOs: 1-66). In some embodiments, species are identified by sequence identity of all or a portion of a 16S rDNA sequence, e.g., at least 90%, 93% 95%, 96%, 97%, 98%, 99%, or 100% identity. Percent identity between a reference and query sequence can be determined using methods known in the art. Non-limiting examples of methods for such determinations are provided below. As used herein, the relatedness between two nucleotide sequences is described by the parameter “identity.”

In one embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical nucleotide in the two aligned sequences on a given position in the alignment and 3) dividing the number of exact matches with the length of the reference sequence.

In another embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical nucleotide in the two aligned sequences on a given position in the alignment and 3) dividing the number of exact matches with the length of the longest of the two sequences.

In another embodiment, the degree of sequence identity between the query sequence and the reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the “alignment length,” where the alignment length is the length of the entire alignment including gaps and overhanging parts of the sequences.

Sequence identity comparisons are, generally, with the aid of a sequence comparison program. These commercially or publicly available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalizing the insertion of gaps, gap extensions and alignment of non-similar amino acids. The scoring system of the comparison algorithms include:

i) assignment of a penalty score each time a gap is inserted (gap penalty score),

ii) assignment of a penalty score each time an existing gap is extended with an extra position (extension penalty score),

iii) assignment of high scores upon alignment of identical amino acids, and

iv) assignment of variable scores upon alignment of non-identical amino acids.

In general, the default values of the alignment program are used for sequence comparisons. Suitable computer programs useful for determining identity include, for example, BLAST (blast.ncbi.nlm.nih.gov).

In an embodiment of the present invention, the alignment program optimizes the alignment over the full-length of selected sequences, e.g., full-length, V4, or V6 16S rDNA sequence. For example, the global alignment program is based on the Needleman-Wunsch algorithm (Needleman and Wunsch, J. Mol. Biol. 48:443-453, 1970). Non-limiting examples of such programs are EMBOSS Needle and EMBOSS Stretcher programs, available at ebi.ac.uk/Tools/psa/.

In one embodiment, the sequences are aligned by a global alignment program and the sequence identity is calculated by identifying the number of exact matches identified by the program divided by the “alignment length,” where the alignment length is the length of the entire alignment including gaps and overhanging parts of the sequences. In a further embodiment, the global alignment program uses the Needleman-Wunsch algorithm and the sequence identity is calculated by identifying the number of exact matches identified by the program divided by the “alignment length,” where the alignment length is the length of the entire alignment including gaps and overhanging parts of the sequences.

In yet a further embodiment, the global alignment program is selected from the group consisting of EMBOSS Needle and EMBOSS stretcher and the sequence identity is calculated by identifying the number of exact matches identified by the program divided by the “alignment length,” where the alignment length is the length of the entire alignment including gaps and overhanging parts of the sequences.

Once the software has produced an alignment, it is possible to calculate percent (%) similarity and percent sequence identity.

In some embodiments, at least one microorganism in a composition can express a tryptophan decarboxylase. In some embodiments, at least one microorganism in the composition can secrete tryptamine. Methods of assaying tryptamine secretion are known in the art (e.g., Williams et al., Cell Host Microbe 16:495-503, 2014). In other embodiments, at least one microorganism in the composition has activity in a GI transit assay (see, e.g., Yano et al., Cell 161:264-276, 2015).

In certain embodiments, a TA composition comprises between 10e2 and 10e12 (e.g., 10e8-10e11 or 10e9-10e10) viable bacteria and/or spores that can express or can be induced to express a tryptophan decarboxylase.

In some embodiments a TA composition can induce an increase in tryptamine levels in a human or non-human animal, e.g., mouse, rat, guinea pig, dog, cat, pig, sheep, or non-human primate.

In general, one or more microorganisms useful in the invention are facultative or obligate anaerobes. In some cases, one or more microorganisms are obligate anaerobes that exhibit aerotolerance. The organisms can be capable or forming spores (herein termed “spore formers”) or non-spore formers. Spore formers may be present in a composition substantially as spores, vegetative cells, or a combination of spores and vegetative cells.

Methods of Assaying Tryptamine

In some embodiments of the invention, the presence and, in some cases, levels of tryptamine are determined. Tryptamine can be assayed using methods known in the art. For example, tryptamine can be assayed using liquid chromatography coupled to tandem mass spectrometry (LC-MS/MS) using a Thermo TSQ Quantum Max triple quadrupole mass spectrometer. Other useful methods include multiple reaction monitoring and information-dependent monitoring (Sridharan et al., Nature Communications 5:5492, 2014).

Tryptamine levels in a subject, e.g., prior to and after administration of a TA composition can be assayed using blood, plasma, serum, stool, urine, or cerebrospinal fluid.

Tryptophan Decarboxylase

In some embodiments, a composition includes at least one bacterial species encoding a tryptophan decarboxylase gene. Methods of identifying such genes are known in the art, for example, see Williams et al. (supra). As described in the Examples, sequence analysis of predicted protein structure and comparison to known microbial tryptophan decarboxylase sequences can be used to identify genes in a bacterial genome. In some cases, the entire sequence of a tryptophan decarboxylase gene is used in the analysis and/or the sequence of the predicted binding site for tryptophan is used.

Treatment of Disease

In embodiments of the invention, a microbiome composition is used to treat or prevent a disease or condition, including ameliorating a sign or symptom of a disease in a subject in need of treatment. In some embodiments, in silico, in vitro, and/or in vivo methods can be used to determine the efficacy of a treatment in altering tryptamine levels in the subject.

Tryptamine is an Ahr agonist. Activation of Ahr reportedly can affect FoxP3 and IL-17 expression and ameliorate colitis, reduce risk of carcinogenesis, and other effects known in the art. Reported tryptamine effects include stimulating ion secretion by intestinal epithelial cells and increasing gut motility (Bhattarai et al., Cell Host Microbe 23(6):775-785, 2018). Tryptamine has also been suggested as an agent for increasing regulatory T cells (Tregs; U.S. Pat. No. 9,028,798). Accordingly, compositions that can increase tryptamine are useful, e.g., for restoring or improving gut homeostasis, treating colitis, treating or preventing intestinal cancer, and/or improving gastrointestinal motility. In general, a TA composition that can increase tryptamine levels is useful for modulating the activity of an aryl hydrocarbon receptor. TA compositions can also be used for increasing Tregs.

Compositions of the invention can also be used to modulate (e.g., increase) 5-hydroxytryptamine, or serotonin. Accordingly, the compositions and methods of the invention can be used in the treatment of diseases and conditions relating to gut motility, gut homeostasis, platelet aggregation, immune responses, and cardiac function, as well as depression, anxiety, irritable bowel syndrome (IBS), cardiovascular disease, and osteoporosis.

Patients

In some embodiments, a subject suitable for treatment with a TA composition is identified as having an abnormal or undesirably low level of tryptamine or 5-hydroxytryptamine, e.g., in feces, blood, plasma, serum, urine, cerebrospinal fluid (CSF), or other body fluid. An effective treatment with a TA composition increases the level of tryptamine or 5-hydroxytryptamine in a selected body fluid, tissue, or feces. Typically, the increased level of tryptamine or 5-hydroxytryptamine is associated with the detectable presence of at least one species from the TA composition in the gastrointestinal tract of the treated subject. In some embodiments, the presence of at least one species or strain of bacterium from the TA composition is detected in the stool of the treated subject. In some embodiments, at least one species or strain of a TA composition can be detected at least 1 day, 2 days, 3 days, 4 days, 5 days, 6 days, 1 week, two weeks, three weeks, 4 weeks, 2 months, 3 months, 4 months, 5 months, 6 months, 8 months, or 1 year after treatment with a TA composition. Such persistence of an administered species (e.g., in the absence or low levels of that species prior to treatment) is termed “engraftment.” In some embodiments, an administered species may not persist or engrafts for a limited period of time; such species may be administered in repeated doses of a TA composition.

A subject treated according to the methods of the invention may have a disease or condition characterized by altered gut motility. In various examples, a subject treated according to the methods of the invention may have a gastrointestinal disease or condition selected from the group consisting of, e.g., irritable bowel syndrome, inflammatory bowel diseases (e.g., infectious colitis, ulcerative colitis, Crohn's disease, ischemic colitis, radiation colitis, and microscopic colitis), and constipation (also see above).

### Definitions

“Synergy” refers to an effect produced by a combination, e.g., of different microbes (for example, different strains, different species, or different clades) that is greater than the expected additive effectives of the combination components. As used herein, “synergy” or “synergistic interactions” refers to the interaction or cooperation of two or more microbes to produce a combined effect greater than the sum of their separate effects. The effect may be based on in vitro or in vivo observations.

A “therapeutically effective amount” of a TA composition described herein can vary according to factors such as the disease state, age, sex, and weight of the individual, and the ability of the compound to elicit a desired response in the individual, e.g., amelioration of at least one disorder parameter, or amelioration of at least one symptom of the disorder (and optionally, the effect of any additional agents being administered). A therapeutically effective amount is also one in which any toxic or detrimental effects of the composition are outweighed by the therapeutically beneficial effects. A composition as described herein is generally administered in a therapeutically effective amount.

The term “microbial engraftment” or “engraftment” refers to the establishment of one or more strains, OTUs or species administered in a therapeutic microbial composition, e.g., a bacterial composition, in a target niche that are absent, undetectable, or present in undesirably low levels in a treated subject prior to treatment. The microbes comprising the engrafted ecology are present in the therapeutic microbial composition and establish as constituents of the subject's microbial ecology. Engrafted OTUs can establish for a transient period of time, or demonstrate long-term stability in the microbial ecology that populates the subject post treatment with a therapeutic microbial composition. Engraftment can be detected through the use of various molecular technologies known in the art, including but not limited to whole metagenome sequencing (WMS), PCR or qPCR with strain- or taxa-specific primers, or with strain- or taxa-specific DNA probes. With whole metagenome sequencing (WMS) data, engraftment can be quantified using strain-specific genomic variants including but not limited to single nucleotide variants (SNVs). With whole metagenome sequencing (WMS) data, engraftment of strains, species, or other taxa can also be quantified based on strain-, species-, or other taxa-specific genomic markers. With whole metagenome sequencing (WMS) data, detection of engraftment can also be aided by whole genome assembly. After such data is generated, engraftment can be assessed statistically at the subject level and/or the population level; at the subject level, the strain- or taxa-specific abundance, relative abundance, or detection must be greater than at baseline or a pre-determined background noise level; at the population level, the strain- or taxa-specific abundance, relative abundance, or detection signal must be greater than a reference placebo arm or a reference background population or dataset.

Limits of detection (LOD) can vary depending upon, for example, the particular sample types under analysis (e.g., stool sample and the water content thereof), the species being detected, and the assays used, as is understood in the art. In various examples, LODs used in the invention range from about 10e5 to 10e7. In one specific example, a LOD used is about 1.75e6 (e.g., 1.75e6).

“Operational taxonomic unit,” “OTU” (or plural, “OTUs”) refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence is the 16S sequence or a portion of the 16S sequence, such as a variable region, e.g., a V4 region. In other embodiments, the entire genomes of two entities are sequenced and compared. In another embodiment, select regions such as multilocus sequence tags (MLST), specific genes, or sets of genes may be genetically compared. In 16S embodiments, OTUs that share ≥97% average nucleotide identity across an entire 16S or a variable region of a 16S sequence are considered the same OTU (see e.g., Claesson et al., Nucleic Acids Res 38:e200, 2010; Konstantinidis et al., Philos Trans R Soc Lond B Biol Sci 361:1929-1940, 2006). In embodiments involving the complete genome, MLSTs, specific genes, or sets of genes, OTUs that share ≥95% average nucleotide identity are considered the same OTU (see e.g. Achtman and Wagner, Nat. Rev. Microbiol. 6:431-440, 2008). OTUs can be distinguished, in some embodiments, by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping”genes), or a combination thereof. Such characterization employs, e.g., WGS data or a whole genome sequence. As used herein, the terms “species” and “OTU” are used interchangeably unless otherwise distinguished by context.

Formulations

TA compositions described herein can be prepared and administered using methods known in the art. In general, compositions are formulated for oral, colonoscopic, or nasogastric delivery although any appropriate method can be used.

A TA formulation can contain one or more pharmaceutical excipients suitable for the preparation of such formulations. In some embodiments, the formulation is a liquid formulation. In some embodiments, a formulation comprising a TA composition can comprise one or more of surfactants, adjuvants, buffers, antioxidants, tonicity adjusters, thickeners or viscosity modifiers and the like.

In some embodiments, treatment includes administering a pharmaceutical TA composition in a formulation that includes a pharmaceutically acceptable carrier. In some embodiments, the excipient includes a capsule or other format suitable for providing the TA composition as an oral dosage form. When an excipient serves as a diluent, it can be a solid, semi-solid, or liquid material, which acts as a vehicle, carrier or medium for the active ingredient. Thus, the formulations can be in the form of tablets, pills, powders, lozenges, sachets, cachets, elixirs, suspensions, emulsions, solutions, syrups, soft or hard capsules, suppositories, or packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, polyethylene glycol, glycerol, and methyl cellulose. The compositions can be formulated so as to provide quick, sustained or delayed release of the active ingredient after administration to the patient by employing procedures known in the art.

A TA composition can be formulated in a unit dosage form. In general, a dosage comprises about 10e2 to 10e9 viable colony forming units (cfu). The term “unit dosage forms” refers to physically discrete units suitable as unitary dosages for human subjects and/or other mammals, each unit containing a predetermined quantity of active material calculated to produce the desired therapeutic effect, in association with a suitable pharmaceutical excipient. A dosage may be administered in multiple delivery vehicles, e.g., multiple pills or capsules.

The amount and frequency of a TA composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The dosage can refer, for example, to the total number of cfus of each individual species or strains; or can refer to the total number of microorganisms in the dose. It is understood in the art that determining the number of organisms in a dosage is not exact and can depend on the method used to determine the number of organisms present. For example, the number of spores in a composition may be determined using a dipicolinic acid assay. In some cases, the number of organisms may be determined using a culture assay. When spores are present, the ability of assays relying on culture methods can depend on efficient germination of spores. Quantitative nucleic acid methods can depend on whether the nucleic acids from non-viable microorganisms are sufficiently reduced or eliminated.

Effective doses can be extrapolated from dose-response curves derived from in vitro or animal model test systems.

### EQUIVALENTS

All technical features can be individually combined in all possible combinations of such features.

The invention may be embodied in other specific forms without departing from the spirit or essential characteristics thereof. The foregoing embodiments are therefore to be considered in all respects illustrative rather than limiting on the invention described herein.

### EXAMPLES

The following non-limiting examples further illustrate embodiments of the inventions described herein.

### Example 1: Genomic and Metabolomic Analysis of Human Feces

Human fecal samples obtained from subjects participating in a Phase II trial of a microbiome composition composed of encapsulated purified eubacterial spores (clinicaltrials.gov; NCT02437487) designed to assess the safety and efficacy of the microbiome composition. Samples were from subjects in the NCT02437487 trial, including samples from both those subjects receiving the treatment and those receiving placebo. Metabolomic analyses of samples for the presence of tryptamine were conducted at Metabolon (Morrisville, N.C.). Metabolomic data included levels of tryptamine per mass of stool (peak AUC, proportional to concentration and assuming linear kinetics of the assay). Samples were also sequenced using whole metagenomics shotgun sequencing and the species name designated based on species-specific genomic markers based on the MetaPhlAn2 database (Truong et al., Nat. Methods 12:902-903, 2015).

Identification of Species-Metabolite Relationships

Standard analysis of paired taxonomic and metabolomic profiles often involves pairwise correlation (e.g., Spearman or Pearson correlation) between species and metabolite abundance to identify species whose abundance is correlated with the abundance of metabolites. This type of correlational analysis typically results in large groups of species being correlated with large groups of metabolites, as has been seen in both cohort and interventional studies. This means that standard correlational analysis does not adequately identify which species are truly mechanistically involved in a selected metabolic function.

To solve this problem, Applicants used a novel approach to identify specific species-metabolite relationships in paired taxonomic and metabolomic profiles. Computational analyses were performed analyzing the relationship between the presence and level of tryptamine and presence of individual bacterial species and combinations of bacterial species. In addition, analyses were performed assessing the relative abundance of a bacterial species and tryptamine.

In the method, a model was developed that can learn to predict the metabolite levels in patient samples from the species profiles measured in those samples. Metabolite levels are predicted based on learning the underlying species-metabolite relationships. Training datasets were developed using a cross-validation approach with methods known in the art.

When a metabolite level is truly determined by a small number of species, this modeling approach recovers a sparse set of species-metabolite relationships. In contrast, standard correlational analysis that is applied to these data recover non-sparse relationships, hindering the ability to identify specific species underlying specific metabolic functions (as is necessary to design therapeutic microbial compositions).

Once the underlying species-metabolite relationships were identified, an assessment was made of a) how well the identified species can predict metabolite levels in vivo, and b) how many species are required to predict in vivo metabolite levels (i.e., if many or few species drive the variation of in vivo metabolite levels). Regarding (a), it was found that some metabolites are well-predicted by species profiles and therefore can likely be modulated by a microbial consortium, whereas other metabolites are not and therefore external or host factors likely predominate. Regarding (b), we found that for some metabolites, the variation of in vivo metabolite levels can be explained by the presence/absence of a small number of species (e.g., 1, 2, 3, 4, 5, 6, 7, 8, 9, 10, 12, 14, 16, 18, or 20 species) whereas for other metabolites, the presence/absence of a large number of species (e.g., >20-50 species) is required to explain in vivo metabolite levels.

Data generated in this manner revealed that tryptamine is a metabolite whose variation is well-predicted by the presence/absence of a small number of species. In vivo tryptamine levels can therefore be manipulated based on therapeutic administration of a small number of species associated with tryptamine levels. Furthermore, because tryptamine levels are affected by few bacterial species, it is likely that only a limited number of bacterial species can be used to target tryptamine with a therapeutic microbial consortium.

### Example 2: Bacterial Species Predictive of In Vivo Tryptamine Levels

The analyses related to tryptamine described in Example 1 identified a small number of species whose presence was related to the presence of tryptamine in a fecal sample. Table 7 provides a ranked list (from most strongly associated with the presence of tryptamine ranked as 1) of the three species most strongly associated with the presence of tryptamine; the three species associated with the highest levels of tryptamine were Ruminococcus gnavus, Lachnospiraceae bacterium 9_1_43BFAA, and Ruminococcus torques.

TABLE 7

| | | Relative in |
|---|---|---|
| | | vivo predictive |
| | Tryptophan | strength |
| Species | Rank | (high to low) |
| Ruminococcus_gnavus | 1 | 1.000000 |
| Lachnospiraceae_bacterium_9_1_43BFAA | 2 | 0.770852 |
| Ruminococcus_torques | 3 | 0.541303 |

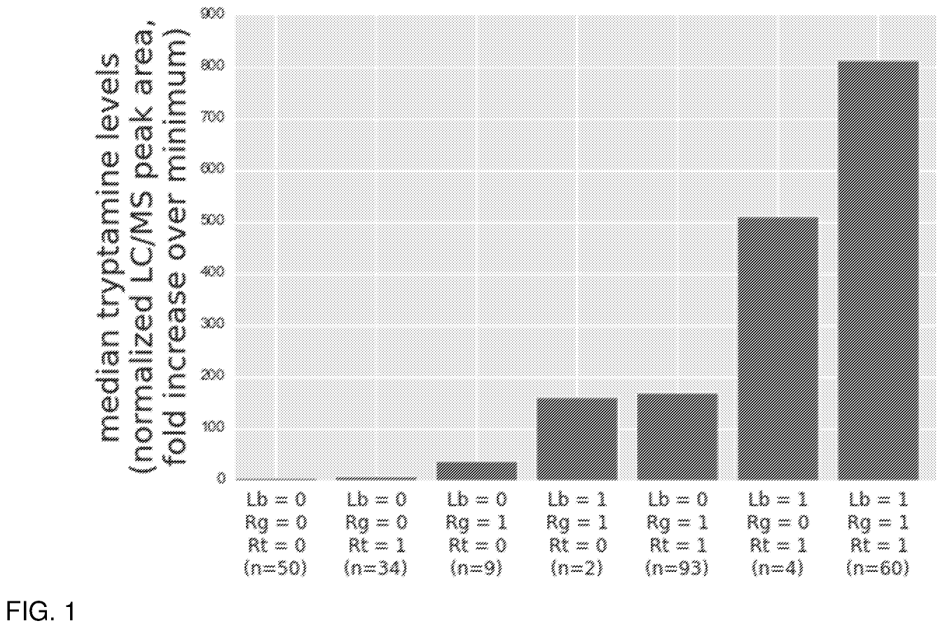

FIG. 1 is an analysis of the three species associated with the highest levels of tryptamine and their relative tryptamine levels in the presence of one, two, or three of the species. The bars represent the median relative levels of tryptamine associated with the indicated species when present in a sample, alone or in various combinations. Although the trend is for higher tryptamine levels in the presence of the greater number of species, the results do not follow a clear additive effect, and are instead synergistic based on the presence of each species. These data indicate that the impact of a species on tryptamine levels is also dependent on the combinatorial presence/absence of other species.

These data demonstrate that such analyses of in vivo data can reveal combinations of bacteria that can affect tryptamine levels in a subject.

Homology Search

In initial studies, previously identified tryptophan decarboxylase genes from Ruminococcus gnavus (uniprot id, A7B VO_RUMGN) and Clostridium sporogenes (uniprot id, J7SZ64_CLOSG) isolates (Williams et al., supra) were used as query sequences to search for homologs in other sequenced bacterial genomes. A database of 1155 human gut-associated bacterial genomes (assembled using WGS information) was assembled to search for homologs with BLASTP; pairs of aligned residues were scored using the BLOSUM62 alignment score matrix; 24 species were identified containing protein homologs with an alignment E-value less than 1e-100 to either query sequence are reported (along with the percent of identical residue matches, alignment length, and e-value; see Table 8); among the 24 homologs identified, the percent of identical residues ranged from 38-99.4% and the alignment lengths ranged from 407-477. These analyses revealed surprising findings.

First, of the top nine species associated with the presence of tryptamine, four of them, including Ruminococcus torques (Tryptamine rank=3) do not harbor a sequence with significant homology to a known bacterial tryptophan decarboxylase. These data indicate that modulation of tryptamine levels may be regulated by additional enzymes and/or mechanisms, besides those related to known tryptophan decarboxylases.

Second, for those species identified as having a sequence with significant homology to a tryptophan decarboxylase, the association of those species with tryptamine levels (see Table 8, Tryptamine rank; lowest rank number reflects the highest association with tryptamine) does not correspond to the percent identity with an “authentic” bacterial tryptophan decarboxylase.

Third, examination of the prevalence of any single species revealed that prevalence was not predictive of Tryptamine rank.

Fourth, while the list of species identified as comprising sequences with homology to tryptophan decarboxylase significantly overlaps with the list of species predictive of tryptamine levels in vivo, there are exceptions. For example, no tryptophan decarboxylase-related sequence homolog was identified in the representative R. torques genome although R. torques is ranked third for in vivo effect on tryptamine levels. Clostridium sporogenes was not found to be predictive of in vivo tryptamine levels despite published data indicating it carries a tryptophan decarboxylase gene.

Fifth, it was found that no sequence had greater than about 30% predicted protein sequence identity over an alignment length of at least 100 residues with a published C. sporogenes tryptophan decarboxylase sequence.

Taken together, Applicants' data demonstrate that species not previously associated with the presence of tryptamine may influence tryptamine production in the gastrointestinal tract and thereby may influence tryptamine levels in other tissues.

TABLE 8

| | % of | | Prevalence of | |
|---|---|---|---|---|
| | identical | Alignment | species in | Tryptamine |
| Species name | matches | Length | samples | rank |
| Ruminococcus_gnavus | 99.37 | 476 | 64% | 1 |
| Clostridium_sporogenes | 93.046 | 417 | 0.8% | — |
| Lachnospiraceae_bacterium_9_1_43BFAA | 76.73 | 477 | 26% | 2 |

Furthermore, these data demonstrate that species prevalence and degree of homology with characterized tryptophan decarboxylase proteins are not related to the strength of the human in vivo relationship of a species with tryptamine levels. Taken together, the data indicate that the mere presence of a tryptophan decarboxylase sequence or even a related sequence is not sufficiently informative to select species that will increase tryptamine levels in a subject, and the analyses provided herein can provide empirical evidence for identification of species affecting tryptamine levels and having use in compositions to increase tryptamine levels in a subject.

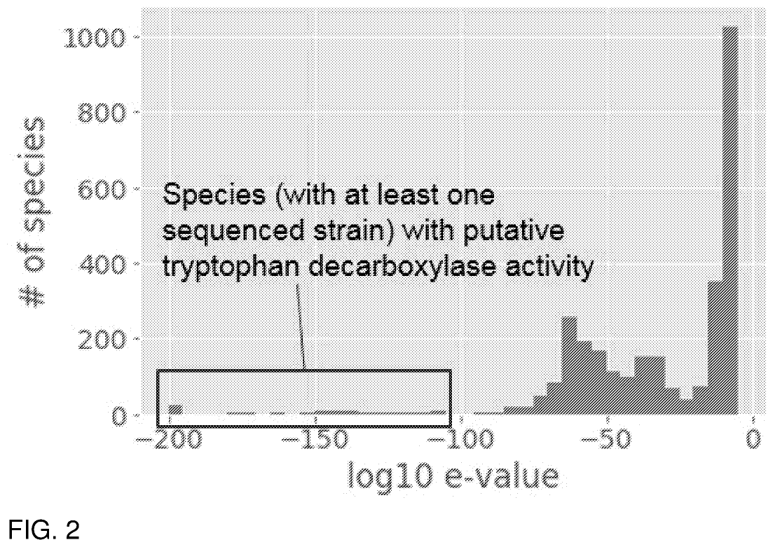

In further studies, Blastp of tryptophan decarboxylase versus a larger bacterial pan-genome database (bitbucket.org/biobakery/humann2/wiki/Home; Huang et al., Nucleic Acids Res. D617-24, 2014) identified putative tryptamine producers (FIG. 2). In these studies, seven species were identified whose presence is predictive of higher tryptamine concentrations across two clinical trials (FIG. 3; Table 9). Five of the seven identified species have strains with identified tryptophan decarboxylase homolog. One species without homolog (Eggerthella unclassified) does not have an associated genome to assess.

TABLE 9

| | | | s004_{—} | s101_{—} | min_blast_{—} | |
|---|---|---|---|---|---|---|
| | s004_avg_{—} | s101_avg_{—} | prev- | prev- | log10_{—} | |
| | coeff | coeff | alance | alance | evalue | blast_hit |
| Ruminococcus_{—}gnavus | 0.396372 | 0.113768 | 0.546075 | 0.473214 | −200.0 | tr\|A7B1V0\|A7B1V0_RUMGN |
| Lachnospiraceae_bacterium_9_1_43BFAA | 0.240586 | 0.023468 | 0.163823 | 0.044643 | −200.0 | tr\|A7B1V0\|A7B1V0_RUMGN |
| Eggerthella_unclassified | 0.118445 | 0.029293 | 0.331058 | 0.416667 | −5.0 | NaN |
| Ruminococcus_{—}gnavus | 0.071891 | 0.016988 | 0.109215 | 0.038690 | −200.0 | tr\|A7B1V0\|A7B1V0_RUMGN |
| Clostridium_{—}nexile | 0.042867 | 0.034824 | 0.286689 | 0.116071 | −200.0 | tr\|A7B1V0\|A7B1V0_RUMGN |
| Lachnospiraceae_bacterium_6_1_63FAA | 0.037267 | 0.037959 | 0.102389 | 0.101190 | −200.0 | tr\|A7B1V0\|A7B1V0_RUMGN |
| Ruminococcus_{—}torques | 0.020212 | 0.050003 | 0.662116 | 0.758929 | −5.0 | NaN |

For Tables 9-12, details regarding the column headings are as follows:

s004_avg_coeff: score indicating how predictive the species is of tryptamine levels in the SERES-004 (C diff) trial.

s101_avg_coeff: score indicating how predictive the species is of tryptamine levels in the SERES-101 (UC) trial.

s004_prevalance: prevalance (fraction of samples) of the speciesn the SERES-004 (C diff) trial.

s101_prevalance: prevalance (fraction of samples) of the speciesin the SERES-101 (UC) trial.

min_blast_log 10_evalue: significance of the homolog to tryptophan decarboxylase (smaller numbers are better, −200 is the smallest [i.e., best hit]). −log 10 of the e-value from blastp.

blast_hit: identifier for the query trypophan decarboxylase used.

Four additional species were identified as being predictive of tryptamine levels in a clinical trial treating C. difficile (Table 10)

TABLE 10

| | | | s004_{—} | s101_{—} | min_blast_{—} | |
|---|---|---|---|---|---|---|
| | s004_avg_{—} | s101_avg_{—} | prev- | prev- | log10_{—} | |
| | coeff | coeff | alance | alance | evalue | blast_hit |
| Lachnospiraceae_bacterium_2_l_58FAA | 0.121176 | 0.000841 | 0.201365 | 0.261905 | −200.0 | tr\|A7B1V0\|A7B1V0_RUMGN |
| Clostridium_{—}aldenense | 0.093945 | 0.003005 | 0.375427 | 0.139881 | −5.0 | NaN |
| Clostridium_{—}citroniae | 0.083953 | 0.000175 | 0.139932 | 0.074405 | −5.0 | NaN |
| Clostridium_{—}clostridioforme | 0.052871 | 0.006717 | 0.416382 | 0.086310 | −5.0 | NaN |

Four additional species were identified as being predictive of tryptamine levels in a clinical trial treating ulcerative colitis (Table 11).

TABLE 11

| | | | s004_{—} | s101_{—} | min_blast_{—} | |
|---|---|---|---|---|---|---|
| | s004_avg_{—} | s101_avg_{—} | prev- | prev- | log10_{—} | |
| | coeff | coeff | alance | alance | evalue | blast_hit |
| Flavonifractor_{—}plautii | −0.003131 | 0.075627 | 0.535836 | 0.535714 | −5.119186 | tr\|J7SZ64\|J7SZ64_CLOSG |
| Veillonella_{—}parvula | −0.018935 | 0.075612 | 0.709898 | 0.428571 | −7.083546 | tr\|A7B1V0\|A7B1V0_RUMGN |
| Blautia_sp_CAG_257 | 0.004683 | 0.072552 | 0.167235 | 0.184524 | −6.401209 | tr\|A7B1V0\|A7B1V0_RUMGN |
| Clostridium_{—}bolteae | 0.002552 | 0.068712 | 0.546075 | 0.404762 | −6.647817 | tr\|A7B1V0\|A7B1V0_RUMGN |

Other species were identified to have tryptophan decarboxylase homology, but were not predictive of tryptamine levels in vivo under the conditions tested (Tables 12 and 13). For these species, the homolog may not have activity on tryptophan as a substrate (Williams et al., Cell Host Microbe 16(4):495-503, 2014) or the species may be lowly prevalent or abundant in vivo.

Table 12 lists species with less than 5% prevalence across both trials (but are detected at least once in either trial). These species may be able to impact in vivo tryptamine levels, but their low prevalence in these studied populations means they may not explain substantial variation in tryptamine in the studied populations. Table 13 lists species with greater than 5% prevalence across both trials. These species do not explain variation in in vivo tryptamine levels despite their substantial prevalence in the studied populations. They may in fact not have activity on tryptophan as a substrate or may show strain variability in activity.

TABLE 12

| | | | s004_{—} | s101_{—} | min_blast_{—} | |
|---|---|---|---|---|---|---|
| | s004_avg_{—} | s101_avg_{—} | prev- | prev- | log10_{—} | |
| | coeff | coeff | alance | alance | evalue | blast_hit |
| Blautia_{—}hansenii | 0.000729 | 0.002454 | 0.040956 | 0.044643 | −200.000000 | tr\|A7B1V0\|A7B1V0_RUMGN |
| Lachnospiraceae_bacterium_2_1_46FAA | 0.000000 | 0.000000 | 0.013652 | 0.000000 | −200.000000 | tr\|A7B1V0\|A7B1V0_RUMGN |
| Coprococcus_sp_HPP0048 | 0.000000 | 0.000000 | 0.003413 | 0.000000 | −200.000000 | tr\|A7B1V0\|A7B1V0_RUMGN |
| Collinsella_{—}tanakaei | 0.000000 | 0.000000 | 0.000000 | 0.014881 | −178.950782 | tr\|A7B1V0\|A7B1V0_RUMGN |
| Clostridium_{—}sporogenes | 0.000000 | 0.000000 | 0.006826 | 0.000000 | −200.000000 | tr\|J7SZ64\|J7SZ64_CLOSG |
| Clostridium_{—}phytofermentans | 0.000000 | 0.000000 | 0.020478 | 0.002976 | −200.000000 | tr\|A7B1V0\|A7B1V0_RUMGN |
| Clostridium_{—}bifermentans | 0.000000 | 0.000000 | 0.006826 | 0.002976 | −200.000000 | tr\|A7B1V0\|A7B1V0_RUMGN |
| Staphylococcus_{—}aureus | 0.000000 | 0.000000 | 0.006826 | 0.000000 | −145.173925 | tr\|A7B1V0\|A7B1V0_RUMGN |

TABLE 13

| | | | s004_{—} | s101_{—} | min_blast_{—} | |
|---|---|---|---|---|---|---|
| | s004_avg_{—} | s101_avg_{—} | prev- | prev- | log10_{—} | |
| | coeff | coeff | alance | alance | evalue | blast_hit |
| Lachnospiraceae_bacterium_4_1_37FAA | 0.025529 | 0.005773 | 0.078498 | 0.041667 | −200.000000 | tr\|A7B1V0\|A7B1V0_RUMGN |
| Clostridium_{—}asparagiforme | 0.008079 | 0.000864 | 0.221843 | 0.142857 | −164.110138 | tr\|A7B1V0\|A7B1V0_RUMGN |
| Clostridium_{—}lavalense_SC43 | 0.002085 | −0.000133 | 0.211604 | 0.071429 | −163.050122 | tr\|A7B1V0\|A7B1V0_RUMGN |
| Holdemania_{—}filiformis | 0.000177 | 0.040108 | 0.344710 | 0.398810 | −200.000000 | tr\|A7B1V0\|A7B1V0_RUMGN |

In additional analyses, consortia were identified that had combinations of greater than one of the seven species listed in Table 1 in ulcerative colitis and C. difficile clinical trials (FIG. 4). These consortia can be used, e.g., in a TA composition, to impact in vivo tryptamine levels more than the single species alone.

### Example 2: Detection of Tryptophan Metabolites in Supernatants

Methods

Applicant tested various bacterial species for the presence of tryptophan metabolites in their supernatants. The presence of the tryptophan metabolites was determined using a colorimetric assay for detection of indolic compounds (Indole Reagent, Anaerobe Systems). Supernatants of selected strains that were identified as producers of Trp metabolites by the colorimetric indole assay were further analyzed by GC-MS to identify the specific metabolites produced.

Results

Several species were shown to produce tryptamine or 5-hydroxytryptamine (5HT). In particular, tryptamine-producing species include Clostridium sporogenes and Ruminococcus gnavus, two of the three species found to correlate with tryptamine presence in human fecal samples (Table 14). 5HT producing species include Bacteroides ovatus and Bacteroides stercoris.

Table 14 is a lists different bacterial species and selected tryptophan metabolites produced by the species. <LOD indicates that the metabolite was below limit of detection. The background metabolite concentration in the bacterial growth media is indicated. The ATCC catalog number is indicated for external strains when applicable.

TABLE 14

| | | LOD | |
|---|---|---|---|
| | | 0.267 | 0.002 |
| | | Compound | |
| | | 5-hydroxytryptamine | |
| | ATCC | (serotonin) | Tryptamine |
| | No. | mM | mM |
| FCM blank | | <LOD | 0.006 |
| Clostridium ghonii | 25757 | <LOD | <LOD |
| Flavonifractor plautii | 29863 | <LOD | <LOD |
| Ruminococcus gnavus strain 1 | 29149 | <LOD | 0.183 |
| Ruminococcus gnavus strain 2 | 39143 | <LOD | 0.173 |
| PY basal blank | | <LOD | 0.013 |
| Bacteroides ovatus | 8483; 700292 | 0.280 | 0.024 |
| Bacteroides stercoris | 43183 | 0.326 | 0.021 |
| Clostridium sporogenes strain 1 | 11437 | <LOD | 0.178 |
| Clostridium sporogenes strain 2 | 7955 | <LOD | 0.337 |
| Ruminococcus gnavus strain 3 | 29149 | <LOD | 0.355 |

Some embodiments of the invention are within the scope of the following numbered paragraphs.

1. A method of altering tryptamine or 5-hydroxytryptamine levels in a subject, the method comprising administering a viable population of at least one bacterial species selected from Table 1, 2, 3, 4, 5, or 6 to the subject.

2. The method of paragraph 1, wherein the levels of tryptamine or 5-hydroxytryptamine in the subject are increased.

3. The method of paragraph 1 or 2, comprising administering a viable population of at least two species of the group consisting of Ruminococcus_gnavus (strain 1), Lachnospiraceae_bacterium_9_1_43BFAA, Eggerthella_unclassified, Ruminococcus_gnavus (strain 2), Clostridium_nexile, Lachnospiraceae_bacterium_6_1_63FAA, and Ruminococcus_torques to the subject.

4. The method of paragraph 1 or 2, comprising administering a viable population of at least two species of the group consisting of Clostridium ghonii, Flavonifractor plautii, Ruminococcus gnavus Bacteroides ovatus, Bacteroides stercoris, and Clostridium sporogenes to the subject.

5. The method of paragraph 1 or 2, comprising administering a viable population of at least two species of the group consisting of Lachnospiraceae_bacterium_2_1_58FAA, Clostridium_aldenense_SC114, Clostridium_citroniae, and Clostridium_clostridioforme to the subject.

6. The method of paragraph 1 or 2, comprising administering a viable population of at least two species of the group consisting of Flavonifractor_plautii, Veillonella_parvula, Blautia_sp_CAG_257_SC146, and Clostridium_bolteae to the subject.

7. The method of paragraph 1 or 2, comprising administering a viable population of at least two species of the group consisting of Blautia_hansenii, Lachnospiraceae_bacterium_2_1_46FAA, Coprococcus_sp_HPP048, Collinsella_tanakaei, Clostridium_sporogenes, Clostridium_phytofermentans, Clostridium_bifermentans, Staphylococcus_aureus, Lachnospiraceae_bacterium_4_1_37FAA, Clostridium_asparagiforme, Clostridium_lavalense_SC43, and Holdemania_filliformis to the subject.

8. The method of any one of paragraphs 3 to 7, comprising administering a viable population of at least 3, 4, 5, 6, 7, 8, 9, 10, 11, or 12 of the listed species to the subject.

9. The method of any one of paragraphs 1 to 8, comprising administering a composition comprising one or more of compositions 1 to 47 of Table 6 to the subject.

10. The method of any one of paragraphs 1 to 9, wherein the subject has a disease or condition characterized by altered gut motility, platelet aggregation, immune response, cardiac function, or bone development.

11. The method of any one of paragraphs 1 to 10, wherein the subject has a disease or condition selected from the group consisting of irritable bowel syndrome, an inflammatory bowel disease, constipation, depression, anxiety, cardiovascular disease, and osteoporosis.

12. The method of paragraph 11, wherein the inflammatory bowel disease is selected from the group consisting of infectious colitis, ulcerative colitis, Crohn's disease, ischemic colitis, radiation colitis, and microscopic colitis.

13. A pharmaceutical formulation comprising a viable population of at least one bacterial species selected from Table 1, 2, 3, 4, 5, or 6.

14. The pharmaceutical formulation of paragraph 13, comprising a viable population of at least two species of the group consisting of Ruminococcus_gnavus (strain 1), Lachnospiraceae_bacterium_9_1_43BFAA, Eggerthella_unclassified, Ruminococcus_gnavus (strain 2), Clostridium_nexile, Lachnospiraceae_bacterium_6_1_63FAA, and Ruminococcus_torques.

15. The pharmaceutical formulation of paragraph 13, comprising a viable population of at least two species of the group consisting of Clostridium ghonii, Flavonifractor plautii, Ruminococcus gnavus Bacteroides ovatus, Bacteroides stercoris, and Clostridium sporogenes.

16. The pharmaceutical formulation of paragraph 13, comprising a viable population of at least two species of the group consisting of Lachnospiraceae_bacterium_2_1_58FAA, Clostridium_aldenense_SC114, Clostridium_citroniae, and Clostridium_clostridioforme.

17. The pharmaceutical formulation of paragraph 13, comprising a viable population of at least two species of the group consisting of Flavonifractor_plautii, Veillonella_parvula, Blautia_sp_CAG_257_SC146, and Clostridium_bolteae.

18. The pharmaceutical formulation of paragraph 13, comprising a viable population of at least two species of the group consisting of Blautia_hansenii, Lachnospiraceae_bacterium_2_1_46FAA, Coprococcus_sp_HPP048, Collinsella_tanakaei, Clostridium_sporogenes, Clostridium_phytofermentans, Clostridium_bifermentans, Staphylococcus_aureus, Lachnospiraceae_bacterium_4_1_37FAA, Clostridium_asparagiforme, Clostridium_lavalense_SC43, and Holdemania_filliformis.

19. The pharmaceutical formulation of any one of paragraphs 14 to 18, comprising a viable population of at least 3, 4, 5, 6, 7, 8, 9, 10, 11, or 12 of the listed species.

20. The pharmaceutical formulation of any one of paragraphs 13 to 19, comprising a composition comprising one or more of compositions 1 to 47 of Table 6.

21. The pharmaceutical formulation of any one of paragraphs 13 to 20, comprising a pharmaceutically acceptable excipient and/or within a capsule (e.g., an enterically coated capsule).

22. A method of altering tryptamine or 5-hydroxytryptamine levels in a subject, the method comprising administering an effective amount of a pharmaceutical formulation of any one of paragraphs 13 to 21 to the subject.

23. The method of paragraph 22, wherein the levels of tryptamine or 5-hydroxytryptamine in the subject are increased.

24. The method of paragraph 22 or 23, wherein the tryptamine or 5-hydroxytryptamine levels are tryptamine or 5-hydroxytryptamine levels in feces of the subject.

25. The method of paragraph 22 or 23, wherein the tryptamine or 5-hydroxytryptamine levels are tryptamine or 5-hydroxytryptamine levels in blood, serum, plasma, urine, or cerebrospinal fluid (CSF) of the subject.

26. A method of treating a subject having a disease or condition characterized by the presence of low tryptamine or 5-hydroxytryptamine levels, the method comprising administering to a subject diagnosed with or at risk for the disease with a therapeutically effective amount of a pharmaceutical formulation of any one of paragraphs 13 to 21.

27. The method of paragraph 26, wherein the subject has a disease or condition characterized by altered gut motility, platelet aggregation, immune response, cardiac function, or bone development.

28. The method of paragraph 26 or 27, wherein the subject has a disease or condition selected from the group consisting of irritable bowel syndrome, an inflammatory bowel disease, constipation, depression, anxiety, cardiovascular disease, and osteoporosis.

29. The method of paragraph 28, wherein the inflammatory bowel disease is selected from the group consisting of infectious colitis, ulcerative colitis, Crohn's disease, ischemic colitis, radiation colitis, and microscopic colitis.

30. A method of increasing the level or activity of regulatory T cells in subject, the method comprising administering a pharmaceutical formulation of any one of paragraphs 13 to 21 to the subject.

31. A method for restoring or improving gut homeostasis, or for preventing or treating intestinal or colon cancer in a subject, the method comprising administering a pharmaceutical formulation of any one of paragraphs 13 to 21 to the subject.

32. A composition comprising at least two different bacterial species that, in combination, can increase tryptamine or 5-hydroxytryptamine levels, as compared to the level of tryptamine or 5-hydroxytryptamine produced by each species alone, (i) in the presence of the same level of tryptophan as the combination, (ii) over a specific period of time in the presence of the same level of tryptophan as the combination, (iii) when administered to an in vivo system, or (iv) when administered in vitro to a model system.

33. The composition of paragraph 32, wherein the composition is selected from the group consisting of a composition comprising the bacterial species of a pharmaceutical formulation of any one of paragraphs 13 to 21.

34. A formulation or composition of any one of paragraphs 13 to 21, 32, or 33, for use in altering the levels of tryptamine or 5-hydroxytryptamine in a subject.

35. The formulation or composition of paragraph 34, wherein the subject has a disease or condition characterized by altered gut motility, platelet aggregation, immune response, cardiac function, or bone development.

36. The formulation or composition of paragraph 34 or 35, wherein the subject has a disease or condition selected from the group consisting of irritable bowel syndrome, an inflammatory bowel disease, constipation, depression, anxiety, cardiovascular disease, and osteoporosis.

37. The formulation or composition of paragraph 36, wherein the inflammatory bowel disease is selected from the group consisting of infectious colitis, ulcerative colitis, Crohn's disease, ischemic colitis, radiation colitis, and microscopic colitis.

38. The formulation or composition of paragraph 36, for use in increasing the level or activity of regulatory T cells in subject.

39. The formulation or composition of paragraph 36, for use in restoring gut homeostasis, or for preventing or treating intestinal or colon cancer in a subject.

Other embodiments are within the scope of the following claims.

What is claimed is:

1. A method of altering tryptamine or 5-hydroxytryptamine levels in a subject, the method comprising administering a viable population of at least one bacterial species selected from Table 1, 2, 3, 4, 5, or 6 to the subject.

2. The method of claim 1, wherein the levels of tryptamine or 5-hydroxytryptamine in the subject are increased.

3. The method of claim 1, comprising administering a viable population of at least two species of the group consisting of Ruminococcus_gnavus (strain 1), Lachnospiraceae_bacterium_9_1_43BFAA, Eggerthella_unclassified, Ruminococcus_gnavus (strain 2), Clostridium_nexile, Lachnospiraceae_bacterium_6_1_63FAA, and Ruminococcus_torques to the subject.

4. The method of claim 1, comprising administering a viable population of at least two species of the group consisting of Clostridium ghonii, Flavonifractor plautii, Ruminococcus gnavus Bacteroides ovatus, Bacteroides stercoris, and Clostridium sporogenes to the subject.

5. The method of claim 1, comprising administering a viable population of at least two species of the group consisting of Lachnospiraceae_bacterium_2_1_58FAA, Clostridium_aldenense_SC114, Clostridium_citroniae, and Clostridium_clostridioforme to the subject.

6. The method of claim 1, comprising administering a viable population of at least two species of the group consisting of Flavonifractor_plautii, Veillonella_parvula, Blautia_sp_CAG_257_SC146, and Clostridium_bolteae to the subject.

7. The method of claim 1, comprising administering a viable population of at least two species of the group consisting of Blautia_hansenii, Lachnospiraceae_bacterium_2_1_46FAA, Coprococcus_sp_HPP048, Collinsella_tanakaei, Clostridium_sporogenes, Clostridium_phytofermentans, Clostridium_bifermentans, Staphylococcus_aureus, Lachnospiraceae_bacterium_4_1_37FAA, Clostridium_asparagiforme, Clostridium_lavalense_SC43, and Holdemania_filliformis to the subject.

8. The method of claim 1, comprising administering a viable population of at least 3, 4, 5, 6, 7, 8, 9, 10, 11, or 12 of the listed species to the subject.

9. The method of claim 1, comprising administering a composition comprising one or more of compositions 1 to 47 of Table 6 to the subject.

10. The method of claim 1, wherein the subject has a disease or condition characterized by altered gut motility, platelet aggregation, immune response, cardiac function, or bone development.

11. The method of claim 1, wherein the subject has a disease or condition selected from the group consisting of irritable bowel syndrome, an inflammatory bowel disease, constipation, depression, anxiety, cardiovascular disease, and osteoporosis.

12. The method of claim 11, wherein the inflammatory bowel disease is selected from the group consisting of infectious colitis, ulcerative colitis, Crohn's disease, ischemic colitis, radiation colitis, and microscopic colitis.

13. A pharmaceutical formulation comprising a viable population of at least one bacterial species selected from Table 1, 2, 3, 4, 5, or 6.

14. The pharmaceutical formulation of claim 13, comprising a viable population of at least two species of the group consisting of Ruminococcus_gnavus (strain 1), Lachnospiraceae_bacterium_9_1_43BFAA, Eggerthella_unclassified, Ruminococcus_gnavus (strain 2), Clostridium_nexile, Lachnospiraceae_bacterium_6_1_63FAA, and Ruminococcus_torques.

15. The pharmaceutical formulation of claim 13, comprising a viable population of at least two species of the group consisting of Clostridium ghonii, Flavonifractor plautii, Ruminococcus gnavus Bacteroides ovatus, Bacteroides stercoris, and Clostridium sporogenes.

16. The pharmaceutical formulation of claim 13, comprising a viable population of at least two species of the group consisting of Lachnospiraceae_bacterium_2_1_58FAA, Clostridium_aldenense_SC114, Clostridium_citroniae, and Clostridium_clostridioforme.

17. The pharmaceutical formulation of claim 13, comprising a viable population of at least two species of the group consisting of Flavonifractor_plautii, Veillonella_parvula, Blautia_sp_CAG_257_SC146, and Clostridium_bolteae.

18. The pharmaceutical formulation of claim 13, comprising a viable population of at least two species of the group consisting of Blautia_hansenii, Lachnospiraceae_bacterium_2_1_46FAA, Coprococcus_sp_HPP048, Collinsella_tanakaei, Clostridium_sporogenes, Clostridium_phytofermentans, Clostridium_bifermentans, Staphylococcus_aureus, Lachnospiraceae_bacterium_4_1_37FAA, Clostridium_asparagiforme, Clostridium_lavalense_SC43, and Holdemania_filliformis.

19. The pharmaceutical formulation of claim 14, comprising a viable population of at least 3, 4, 5, 6, 7, 8, 9, 10, 11, or 12 of the listed species.

20. The pharmaceutical formulation of claim 13, comprising a composition comprising one or more of compositions 1 to 47 of Table 6.

21. The pharmaceutical formulation of claim 13, comprising a pharmaceutically acceptable excipient or comprised within a capsule.

22. A method of altering tryptamine or 5-hydroxytryptamine levels in a subject, the method comprising administering an effective amount of a pharmaceutical formulation of claim 13 to the subject.

23. The method of claim 22, wherein the levels of tryptamine or 5-hydroxytryptamine in the subject are increased.

24. The method of claim 22, wherein the tryptamine or 5-hydroxytryptamine levels are tryptamine or 5-hydroxytryptamine levels in feces of the subject.

25. The method of claim 22, wherein the tryptamine or 5-hydroxytryptamine levels are tryptamine or 5-hydroxytryptamine levels in blood, serum, plasma, urine, or cerebrospinal fluid (CSF) of the subject.

26. A method of treating a subject having a disease or condition characterized by the presence of low tryptamine or 5-hydroxytryptamine levels, the method comprising administering to a subject diagnosed with or at risk for the disease with a therapeutically effective amount of a pharmaceutical formulation of claim 13.

27. The method of claim 26, wherein the subject has a disease or condition characterized by altered gut motility, platelet aggregation, immune response, cardiac function, or bone development.

28. The method of claim 26, wherein the subject has a disease or condition selected from the group consisting of irritable bowel syndrome, an inflammatory bowel disease, constipation, depression, anxiety, cardiovascular disease, and osteoporosis.

29. The method of claim 28, wherein the inflammatory bowel disease is selected from the group consisting of infectious colitis, ulcerative colitis, Crohn's disease, ischemic colitis, radiation colitis, and microscopic colitis.

30. A method of increasing the level or activity of regulatory T cells in subject, the method comprising administering a pharmaceutical formulation of claim 13 to the subject.

31. A method for restoring or improving gut homeostasis, or for preventing or treating intestinal or colon cancer in a subject, the method comprising administering a pharmaceutical formulation of claim 13 to the subject.

32. A composition comprising at least two different bacterial species that, in combination, can increase tryptamine or 5-hydroxytryptamine levels, as compared to the level of tryptamine or 5-hydroxytryptamine produced by each species alone, (i) in the presence of the same level of tryptophan as the combination, (ii) over a specific period of time in the presence of the same level of tryptophan as the combination, (iii) when administered to an in vivo system, or (iv) when administered in vitro to a model system.

33. The composition of claim 32, wherein the composition is a composition including a bacterial species of a pharmaceutical formulation of claim 13.

34. A formulation or composition of claim 13, for use in altering the levels of tryptamine or 5-hydroxytryptamine in a subject.

35. The formulation or composition of claim 34, wherein the subject has a disease or condition characterized by altered gut motility, platelet aggregation, immune response, cardiac function, or bone development.

36. The formulation or composition of claim 34, wherein the subject has a disease or condition selected from the group consisting of irritable bowel syndrome, an inflammatory bowel disease, constipation, depression, anxiety, cardiovascular disease, and osteoporosis.

37. The formulation or composition of claim 36, wherein the inflammatory bowel disease is selected from the group consisting of infectious colitis, ulcerative colitis, Crohn's disease, ischemic colitis, radiation colitis, and microscopic colitis.

38. The formulation or composition of claim 36, for use in increasing the level or activity of regulatory T cells in subject.

39. The formulation or composition of claim 36, for use in restoring gut homeostasis, or for preventing or treating intestinal or colon cancer in a subject.


SEQUENCE LISTING (75 sequences)

SEQ ID NO: 1 - DNA, 1160 bp, Blautia hansenii
tcagagagtttgatcctggctcaggatgaacgctggcggcgtgcttaacacatgcaagtcgagcgaagcacttatcattgactcttcggaagatttgatatttgactgagcggcggacgggtgagtaacgcgtgggtaacctgcctcatacaggggaataacagttagaaatggctgctaatgccgcataagcgcacaggatcgcatggtctggtgtgaaaaactgaggtggtatgagatggacccgcgtctgattaggtagttggtggggtaacggcctaccaagccgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggtgcaaagcagttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatctgcctgaccgttccttgaccggagctttccttcgggacaggcaagacaggtggtgcatggttgtcgtcagctcgtgtcgtgagatgttgggttaagtcccgcaacgagcgcaacccctatccttagtagccagcagttcggctgggcactctagggagactgccggggataacccg

SEQ ID NO: 2 - DNA, 802 bp, Blautia hansenii
ggacggtaactgacgttgaggctcgaaagcgtggggagcaaacaggattagataccctggtagtccacgccgtaaacgatgaatactaggtgtcggggtgcaaagcagttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatctgcctgaccgttccttaaccggagctttccttcgggacaggcaagacaggtggtgcatggttgtcgtcagctcgtgtcgtgagatgttgggttaagtcccgcaacgagcgcaacccctatccttagtagccagcagtccggctgggcactctagggagactgccggggataacccggaggaaggcggggacgacgtcaaatcatcatgccccttatgatttgggctacacacgtgctacaatggcgtaaacaaagggaagcgaagcggtgacgcttagcaaatctcaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttatggagggagctgccgaaggcgggaccgataactggggtgaagtcgtaacaaggtagccgtatcggaaggtgcggctggatcacctccttt

SEQ ID NO: 3 - DNA, 1529 bp, Blautia caecimuris
acagagagtttgatcctggctcaggatgaacgctggcggcgtgcttaacacatgcaagtcgaacggggattattttgacagagacttcggttgaagtcgttataatcctagtggcggacgggtgagtaacgcgtgggtaacctgcctcacactgggggataacagtcagaaatgactgctaataccgcataagcgcacagagctgcatggctcagtgtgaaaaactccggtggtgtgagatggacccgcgttggattagccagttggcagggtaacggcctaccaaagcgacgatccatagccggcctgagagggtggacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggagcagcaagtctgatgtgaaaggcgggggctcaacccccggactgcattggaaactgttgatcttgagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcagagccattcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccctctgaccggtgagtaacgtcacctttccttcgggacagaggagacaggtggtgcatggttgtcgtcagctcgtgtcgtgagatgttgggttaagtcccgcaacgagcgcaacccctatccttagtagccagcggtttggccgggcactctgaggagactgccagggataacctggaggaaggcggggatgacgtcaaatcatcatgccccttatgatttgggctacacacgtgctacaatggcgtaaacaaagggaagcgagagtgtgagcttaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgaccgaaccgaaaggacggagctgccgaaggcgggacggatgactggggtgaagtcgtaacaaggtagccgtatcggaaggtgcggctggatcacctccttt

SEQ ID NO: 4 - DNA, 1531 bp, Clostridium aldenense
tttgagagtttgatcctggctcaggatgaacgctggcggcgtgcctaacacatgcaagtcgaacgaagcgattaggatgaagttttcggatggattcctggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcggaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgctgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggcgcgtaacggtgcccttccttcgggacaggggagacaggtggtgcatggttgtcgtcagctcgtgtcgtgagatgttgggttaagtcccgcaacgagcgcaacccttatccttagtagccagcacgtcgtggtgggcactctagggagactgccagggataacctggaggaaggtggggatgacgtcaaatcatcatgccccttatgatttgggctacacacgtgctacaatggcgtaaacaaagggaggcgaccctgcgaaggtaagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccttaacaggagggagctgccgaaggcggggcaggtaactggggtgaagtcgtaacaaggtagccgtatcggaaggtgcggctggatcacctccttt

SEQ ID NO: 5 - DNA, 1527 bp, Clostridium asparagiforme
tttgagagtttgatcctggctcaggatgaacgctggcggcgtgcctaacacatgcaagtcgaacgaagcattttagatgaagttttcggatggattctgagatgactgagtggcggacgggtgagtaacacgtggataacctgcctcacactgggggacaacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcactccacctggggagtacgttcgcaagaatgaaactcaaaggaattgacggggacccgcacaagcggtggagcatgtggtttaattcgaagcaacgcgaagaaccttaccaagtcttgacatccccctgaccggcgtgtaacggcgcctttccttcgggacaggggagacaggtggtgcatggttgtcgtcagctcgtgtcgtgagatgttgggttaagtcccgcaacgagcgcaacccttatccttagtagccagcattaagatgggcactctagggagactgccagggacaacctggaggaaggtggggatgacgtcaaatcatcatgccccttatgatttgggctacacacgtgctacaatggcgtaaacaaagggaagcgaccctgcgaaggtgagcaaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgtatcggaaggtgcggctggatcacctccttt

SEQ ID NO: 6 - DNA, 1499 bp, Clostridium bolteae
tttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatatggaagtcgcatgacttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacgggggacccgcacaagtagcggagcatgtggtttaattcgaagcaacgcgaagaaccttacctaagcttgacatcccactgacctctccctaatcggagattcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaaaccaatgactggggtgaagtcgtaacaaggtagccgtatcggaaggtgcggctggatcacctccttt

SEQ ID NO: 7 - DNA, 1499 bp, Clostridium bifermentans
tttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatatgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtaggggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaaaccaatgactggggtgaagtcgtaacaaggtagccgtatcggaaggtgcggctggatcacctccttt

SEQ ID NO: 8 - DNA, 926 bp, Clostridium bifermentans
atttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatacggaagtcgcatgacttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggtttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttaccccctcggtgccgcagctaacgcattaagtactccgcctgggaagtacgctcgcaagagtgaaactcaaaggaattgacggggacccgcacaagtagcggagcatgtggtttaattcgaagcaacgc

SEQ ID NO: 9 - DNA, 1497 bp, Clostridium bifermentans
tttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataatatatgaaagtcgcatggcttttatatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaagctctgtcctcaaggaagataatgacggtacttgaggaggaagccccggctaactacgtgccagcagccgcggtaatacgtagggggctagcgttatccggaattactgggcgtaaagggtgcgtaggtggtttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaaaccaatgactggggtgaagtcgtaacaaggtagccgtatcggaaggtgcggctggatcacctccttt

SEQ ID NO: 10 - DNA, 1503 bp, Clostridium bifermentans
tttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatatgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggtttttcaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtcgccgcagctaacgcattaagtactccgcctgggaagtacgctcgcaagagtagaaactcaaaggaattgacggggacccggccacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaaaccaatgactggggtgaagtcgtaacaaggtagccgtatcggaaggtgcggctggatcacctccttt

SEQ ID NO: 11 - DNA, 1317 bp, Clostridium bifermentans
tttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatatgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtactttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatca

SEQ ID NO: 12 - DNA, 957 bp, Clostridium bifermentans
tttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataatatatgaaagtcgcatggcttttatatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgttgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccac

SEQ ID NO: 13 - DNA, 1499 bp, Clostridium bifermentans
tttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatacgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacggggggcagcagtggggaatattgcacaatgggcgaaagactgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaaaccaatgactggggtgaagtcgtaacaaggtagccgtatcggaaggtgcggctggatcacctccttt

SEQ ID NO: 14 - DNA, 895 bp, Clostridium bifermentans
atttgagagtttgatcctggctcaggatgaacgctggcggcgtgcctaacacatgcaagtcgagcgatctcttcggagagagcggcggacgggtgagtaacgcgtgggtaacctgccctgtacacacggataatataccgaaaggtatactaatacgggataacatacgaaagtcgcatggcttttatatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaag

SEQ ID NO: 15 - DNA, 1498 bp, Clostridium bifermentans
tttgagagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataatatatgaaagtcgcatggcttttatatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggaccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctggaactcgcctacatgaagctggagttactagtaatcgcagatcagaatgctgcggtgaatgcgttcccgggtcttgtacacaccgcccgtcacaccatggaagttggggcgcccgaagccggtttagctaaccttttaggaagcggccgtcgaaggtgaaaccaatgactggggtgaagtcgtaacaaggtagccgtatcggaaggtgcggctggatcacctccttt

SEQ ID NO: 16 - DNA, 1528 bp, Clostridium bolteae
atgagagtttgatcctggctcaggatgaacgctggcggcgtgcctaacacatgcaagtcgaacgaagcaattgaaggaagttttcggatggaattcgattgactgagtggcggacgggtgagtaacgcgtggataacctgcctcacactgggggataacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 17 - DNA, 1426 bp, Clostridium citroniae
  misc_feature (1422)..(1426): N is A, T, C, or G
tttgagagtttgatcctggctcaggatgaacgctggcggcgtgcctaacacatgcaagtcgaacgaagcaatcagaatgaaattttcggatggatttctggttgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaannnnn

SEQ ID NO: 18 - DNA, 1528 bp, Clostridium clostridioforme
atgagagtttgatcctggctcaggatgaacgctggcggcgtgcctaacacatgcaagtcgaacgaagcaattaaaggaagttttcggacggaatttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccttttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggcggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 19 - DNA, 1528 bp, Clostridium clostridioforme
atgagagtttgatcctggctcaggatgaacgctggcggcgtgcctaacacatgcaagtcgaacgaagcaattaaaggaagttttcggatggaatttgattgactgagtggcggacgggtgagtaacgcgtggataacctgcctcacactgggggataacagttagaaatgactgctaataccgcataagcgcacagtgccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 20 - DNA, 1528 bp, Clostridium clostridioforme
atgagagtttgatcctggctcaggatgaacgctggcggcgtgcctaacacatgcaagtcgaacgaagcaattaaaggaagttttcggatggaatttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttggccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 21 - DNA, 1529 bp, Clostridium lavalense
tttgagagtttgatcctggctcaggatgaacgctggcggcgtgcctaacacatgcaagtcgaacgaagcatttcagatgaagttttcggatggattctgagatgactgagtggcggacgggtgagtaacacgtggataacctgcctcacactgggggacaacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcactccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgcctttccttcgggacaagggagacaggtggtgcatggttgtcgtcagctcgtgtcgtgagatgttgggttaagtcccgcaacgagcgcaacccttatccttagtagccagcattaagatgggcactctagggagactgccagggacaacctggaggaaggtggggatgacgtcaaatcatcatgccccttatgatttgggctacacacgtgctacaatggcgtaaacaaagggaagcgaccctgcgaaggtgagcaaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 22 - DNA, 1524 bp, Clostridium nexile
  misc_feature (1)..(7): N is A, T, C, or G
  misc_feature (191)..(192): N is A, T, C, or G
  misc_feature (348)..(348): N is A, T, C, or G
  misc_feature (437)..(437): N is A, T, C, or G
nnnnnnngagatttgatcctggctcaggatgaacgctggccggccgtgcttacacatgcagtcgaacgaagcgcttaaactggatttcttcggattgaagtttttgctgactgagtggcggacgggtgagtaacgcgtgggtaacctgcctcatacagggggataacagttagaaatgactgctaataccnnataagcgcacagtgctgcatggcacagtgtaaaaactccggtggtatgagatggacccgcgtctgattagctagttggtggggtaacggcctaccaaggcgacgatcagtagccggcctgagagggtgaacggccacattgggactgagacacggnccaaactcctacgggaggcagcagtggggaatattgcacaatgggggaaaccctgatgcagcgacgccgcgtgagcgaagaagtatttnggtatgtaaagctctatcagcagggaagaaaatgacggtacctgactaagaagcaccggctaaatacgtgccagcagccgcggtaatacgtaggtgcaagcgttatccggatttactgggtgtaaagggagcgtagacggttgtgtaagtctgatgtgaaagcccggggctcaaccccggactgcattggaaactatgtaactagagtgtcggagaggtaagcggaattcctagtgtagcggtgaaatgcgtagatattaggaggaacaccagtggcgaaggcggcttactggacgatcactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcgtggagcatgtggtttaattcgagcaacgcgaagaccttacctggtcttgacatcccggtgaccggtccagtaatgggacctttccttcgggacacggtgacaggtggtgcatggttgtcgtcagctcgtgtcgtgagatgttgggttaagtcccgcaacgagcgcaacccctatcttcagtagccagcatttaaggtgggcactctggagagactgccagggataacctggaggaaggtggggatgacgtcaaatcatcatgccccttatgaccagggctacacacgtgctacaatggcgtaaacaaagggaagcgaacctgtgaggggaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctcc

SEQ ID NO: 23 - DNA, 1527 bp, Clostridium phytofermentans
tttgagagtttgatcctggctcaggatgaacgctggcggcgtgcttaacacatgcaagtcgaacggagtttttaaatggaagtcctcggatggaagtttagaaacttagtggcggacgggtgagtaacgcgtgggtaacctgcctcatacagggggataacagtcggaaacgattgctaaaaccgcataacatagcgaaaccgcatgattttgctatcaaatatttataggtatgagatggacccgcgtctgattagctagttggtggggtaatggcctaccaaggcgacgatcagtagccggcttgagagagtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagataatgacagtacctgactaagaagccccggctaactacgtgccagcagccgcggtaatacgtagggggcaagcgttatccggatttactgggtgtaaagggagcgtaggtggtaggtcaagtcagatgtgaaagcccagggctcaaccctgggactgcatttgaaactggcttactagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactagctgtcggggctatcatggcttcggtggcgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgacaaccgagtaacgtcggtcttccttcgggacagaggtgacaggtggtgcatggttgtcgtcagctcgtgtcgtgagatgttgggttaagtcccgcaacgagcgcaacccctatctttagtagccagcagttcggctgggcactctagagagactgccagggataacctggaggaaggcggggatgacgtcaaatcatcatgccccttatgatttgggctacacacgtgctacaatggtgactacaaagagaagcaagcctgcgagggggagcaaatctcaaaaaggtcatcccagttcggattgtactctgcaactcgagtacatgaagctggaatcgctagtaatcgcgaatcagaatgtcgcggtgaatacgttcccgggtcttgtacacaccgcccgtcacaccatgggagtaggtaacgcccgaagtcagtgacccaaccgtaaggagggagctgccgaaggcgggatctataactggggtgaagtcgtaacaaggtagccgtatcggaaggtgcggctggatcacctccttt

SEQ ID NO: 24 - DNA, 1528 bp, Clostridium phytofermentans
tttgagagtttgatcctggctcaggatgaacgctggcggcgtgcttaacacatgcaagtcgaacggagtttttaaatggaagccctcgggtggaagtttagaaacttagtggcggacgggtgagtaacgcgtgggtaacctgcctcatacagggggataacagtcggaaacgattgctaaaaccgcataatatagcgaaaccgcatgattttgctatcaaatatttataggtatgagatgggcccgcgtctgattagctagttggtggggtaatggcctaccaaggcgacgatcagtagccggcttgagagagtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagataatgacagtacctgactaagaagccccggctaactacgtgccagcagccgcggtaatacgtagggggcaagcgttatccggatttactgggtgtaaagggagcgtaggtggtaggtcaagtcagatgtgaaagcccagggctcaaccctgggactgcatttgaaactggcttactagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactagctgttggggtccgataggacttcggtggcgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgacaaccgagtaacgtcggtcttccttcgggacagaggtgacaggtggtgcatggttgtcgtcagctcgtgtcgtgagatgttgggttaagtcccgcaacgagcgcaacccctatctttagtagccagcagttcggctgggcactctagagagactgccagggataacctggaggaaggcggggatgacgtcaaatcatcatgccccttatgatttgggctacacacgtgctacaatggtgactacaaagagaagcaagcctgcgagggggagcaaatctcaaaaaggtcatcccagttcggattgtactctgcaactcgagtacatgaagctggaatcgctagtaatcgcgaatcagaatgtcgcggtgaatacgttcccgggtcttgtacacaccgcccgtcacaccatgggagtaggtaacgcccgaagtcagtgacccaaccgtaaggagggagctgccgaaggcgggatctataactggggtgaagtcgtaacaaggtagccgtatcggaaggtgcggctggatcacctccttt

SEQ ID NO: 25 - DNA, 1528 bp, Clostridium phytofermentans; identical to SEQ ID NO: 24

SEQ ID NO: 26 - DNA, 1527 bp, Clostridium phytofermentans
tttgagagtttgatcctggctcaggatgaacgctggcggcgtgcttaacacatgcaagtcgaacggagtttttaaatggaagtcctcggatggaagtttagaaacttagtggcggacgggtgagtaacgcgtgggtaacctgcctcatacagggggataacagtcggaaacgattgctaaaaccgcataatatagcgaaaccgcatgattttgctatcaaatatttataggtatgagatgggcccgcgtctgattagctagttggtggggtaatggcctaccaaggcgacgatcagtagccggcttgagagagtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagataatgacagtacctgactaagaagccccggctaactacgtgccagcagccgcggtaatacgtagggggcaagcgttatccggatttactgggtgtaaagggagcgtaggtggtaggtcaagtcagatgtgaaagcccagggctcaaccctgggactgcatttgaaactggcttactagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactagctgtcggggctatcatggcttcggtggcgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgacaaccgagtaacgtcggtcttccttcgggacagaggtgacaggtggtgcatggttgtcgtcagctcgtgtcgtgagatgttgggttaagtcccgcaacgagcgcaacccctatctttagtagccagcagttcggctgggcactctagagagactgccagggataacctggaggaaggcggggatgacgtcaaatcatcatgccccttatgatttgggctacacacgtgctacaatggtgactacaaagagaagcaagcctgcgagggggagcaaatctcaaaaaggtcatcccagttcggattgtactctgcaactcgagtacatgaagctggaatcgctagtaatcgcgaatcagaatgtcgcggtgaatacgttcccgggtcttgtacacaccgcccgtcacaccatgggagtaggtaacgcccgaagtcagtgacccaaccgtaaggagggagctgccgaaggcgggatctataactggggtgaagtcgtaacaaggtagccgtatcggaaggtgcggctggatcacctccttt

SEQ ID NO: 27 - DNA, 1527 bp, Clostridium phytofermentans
tttgagagtttgatcctggctcaggatgaacgctggcggcgtgcttaacacatgcaagtcgaacggagtttttaaatggaagtcctcggatggaagtttagaaacttagtggcggacgggtgagtaacgcgtgggtaacctgcctcatacagggggataacagtcggaaacgattgctaaaaccgcataatatagcgaaaccgcatgattttgctatcaaatatttataggtatgagatgggcccgcgtctgattagctagttggtggggtaatggcctaccaaggcgacgatcagtagccggcttgagagagtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagataatgacagtacctgactaagaagccccggctaactacgtgccagcagccgcggtaatacgtagggggcaagcgttatccggatttactgggtgtaaagggagcgtaggtggtaggtcaagtcagatgtgaaagcccagggctcaaccctgggactgcatttgaaactggcttactagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactagctgtcggggctatcatggcttcggtggcgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgacaaccgagtaacgtcggtcttccttcgggacagaggtgacaggtggtgcatggttgtcgtcagctcgtgtcgtgagatgttgggttaagtcccgcaacgagcgcaacccctatctttagtagccagcagttcggctgggcactctagagagactgccagggataacctggaggaaggcggggatgacgtcaaatcatcatgccccttatgatttgggctacacacgtgctacaatggtgactacaaagagaagcaagcctgcgagggggagcaaatctcaaaaaggtcatcccagttcggattgtactctgcaactcgagtacatgaagctggaatcgctagtaatcgcgaatcagaatgtcgcggtgaatacgttcccgggtcttgtacacaccgcccgtcacaccatgggagtaggtaacgcccgaagtcagtgacccaaccgtaaggagggagctgccgaaggcgggatttataactggggtgaagtcgtaacaaggtagccgtatcggaaggtgcggctggatcacctccttt

SEQ ID NO: 28 - DNA, 1527 bp, Clostridium phytofermentans
tttgagagtttgatcctggctcaggatgaacgctggcggcgtgcttaacacatgcaagtcgaacggagtttttaaatggaagtcctcggatggaagtttagaaacttagtggcggacgggtgagtaacgcgtgggtaacctgcctcatacagggggataacagtcggaaacgattgctaaaaccgcataacatagcgaaaccgcatgattttgctatcaaatatttataggtatgagatgggcccgcgtctgattagctagttggtggggtaatggcctaccaaggcgacgatcagtagccggcttgagagagtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagataatgacagtacctgactaagaagccccggctaactacgtgccagcagccgcggtaatacgtagggggcaagcgttatccggatttactgggtgtaaagggagcgtaggtggtaggtcaagtcagatgtgaaagcccagggctcaaccctgggactgcatttgaaactggcttactagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactagctgtcggggctatcatggcttcggtggcgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgacaaccgagtaacgtcggtcttccttcgggacagaggtgacaggtggtgcatggttgtcgtcagctcgtgtcgtgagatgttgggttaagtcccgcaacgagcgcaacccctatctttagtagccagcagttcggctgggcactctagagagactgccagggataacctggaggaaggcggggatgacgtcaaatcatcatgccccttatgatttgggctacacacgtgctacaatggtgactacaaagagaagcaagcctgcgagggggagcaaatctcaaaaaggtcatcccagttcggattgtactctgcaactcgagtacatgaagctggaatcgctagtaatcgcgaatcagaatgtcgcggtgaatacgttcccgggtcttgtacacaccgcccgtcacaccatgggagtaggtaacgcccgaagtcagtgacccaaccgtaaggagggagctgccgaaggcgggatttataactggggtgaagtcgtaacaaggtagccgtatcggaaggtgcggctggatcacctccttt

SEQ ID NO: 29 - DNA, 1527 bp, Clostridium phytofermentans
tttgagagtttgatcctggctcaggatgaacgctggcggcgtgcttaacacatgcaagtcgaacggagtttttaaatggaagccctcgggtggaagtttagaaacttagtggcggacgggtgagtaacgcgtgggtaacctgcctcatacagggggataacagtcggaaacgattgctaaaaccgcataatatagcgaaaccgcatgattttgctatcaaatatttataggtatgagatgggcccgcgtctgattagctagttggtggggtaatggcctaccaaggcgacgatcagtagccggcttgagagagtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagataatgacagtacctgactaagaagccccggctaactacgtgccagcagccgcggtaatacgtagggggcaagcgttatccggatttactgggtgtaaagggagcgtaggtggtaggtcaagtcagatgtgaaagcccagggctcaaccctgggactgcatttgaaactggcttactagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactagctgtcggggctatcatggcttcggtggcgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgacaaccgagtaacgtcggtcttccttcgggacagaggtgacaggtggtgcatggttgtcgtcagctcgtgtcgtgagatgttgggttaagtcccgcaacgagcgcaacccctatctttagtagccagcagttcggctgggcactctagagagactgccagggataacctggaggaaggcggggatgacgtcaaatcatcatgccccttatgatttgggctacacacgtgctacaatggtgactacaaagagaagcaagcctgcgagggggagcaaatctcaaaaaggtcatcccagttcggattgtactctgcaactcgagtacatgaagctggaatcgctagtaatcgcgaatcagaatgtcgcggtgaatacgttcccgggtcttgtacacaccgcccgtcacaccatgggagtaggtaacgcccgaagtcagtgacccaaccgtaaggagggagctgccgaaggcgggatttataactggggtgaagtcgtaacaaggtagccgtatcggaaggtgcggctggatcacctccttt

SEQ ID NO: 30 - DNA, 1527 bp, Clostridium phytofermentans
tttgagagtttgatcctggctcaggatgaacgctggcggcgtgcttaacacatgcaagtcgaacggagtttttaaatggaagtcctcggatggaagtttagaaacttagtggcggacgggtgagtaacgcgtgggtaacctgcctcatacagggggataacagtcggaaacgattgctaaaaccgcataacatagcgaaaccgcatgattttgctatcaaatatttataggtatgagatgggcccgcgtctgattagctagttggtggggtaatggcctaccaaggcgacgatcagtagccggcttgagagagtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagataatgacagtacctgactaagaagccccggctaactacgtgccagcagccgcggtaatacgtagggggcaagcgttatccggatttactgggtgtaaagggagcgtaggtggtaggtcaagtcagatgtgaaagcccagggctcaaccctgggactgcatttgaaactggcttactagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactagctgtcggggctatcatggcttcggtggcgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgacaaccgagtaacgtcggtcttccttcgggacagaggtgacaggtggtgcatggttgtcgtcagctcgtgtcgtgagatgttgggttaagtcccgcaacgagcgcaacccctatctttagtagccagcagttcggctgggcactctagagagactgccagggataacctggaggaaggcggggatgacgtcaaatcatcatgccccttatgatttgggctacacacgtgctacaatggtgactacaaagagaagcaagcctgcgagggggagcaaatctcaaaaaggtcatcccagttcggattgtactctgcaactcgagtacatgaagctggaatcgctagtaatcgcgaatcagaatgtcgcggtgaatacgttcccgggtcttgtacacaccgcccgtcacaccatgggagtaggtaacgcccgaagtcagtgacccaaccgtaaggagggagctgccgaaggcgggatctataactggggtgaagtcgtaacaaggtagccgtatcggaaggtgcggctggatcacctccttt

SEQ ID NO: 31 - DNA, 1509 bp, Clostridium sporogenes
attgagagtttgatcctggctcaggacgaacgctggcggcgtgcttaacacatgcaagtcgagcgatgaagcttccttcgggaagtggattagcggcggacgggtgagtaacacgtgggtaacctgcctcaaagtgggggatagccttccgaaaggaagattaataccgcataatataagagaatcgcatgatttttttatcaaagatttattgctttgagatggacccgcggcgcattagctagttggtaaggtaacggcttaccaaggcaacgatgcgtagccgacctgagagggtgatcggccacattggaactgagacacggtccagactcctacgggaggcagcagtggggaatattgcgcaatgggggaaaccctgacgcagcaacgccgcgtgggtgatgaaggtcttcggattgtaaagccctgttttctgggacgataatgacggtaccagaggaggaagccacggctaactacgtgccagcagccgcggtaatacgtaggtggcgagcgttgtccggatttactgggcgtaaagggtgcgtaggcggatgtttaagtgggatgtgaaatccccgggcttaacctgggggctgcattccaaactggatatctagagtgcaggagaggaaagcggaattcctagtgtagcggtgaaatgcgtagagattaggaagaacaccagtggcgaaggcggctttctggactgtaactgacgctgaggcacgaaagcgtgggtagcaaacaggattagataccctggtagtccacgccgtaaacgatggatactaggtgtagggggtatcaactccccctgtgccgcagttaacacaataagtatcccgcctggggagtacggtcgcaagattaaaactcaaaggaattgacgggggcccgcacaagcagcggagcatgtggtttaattcgaagcaacgcgaagaaccttacctggacttgacatcccttgcatagcctagagataggtgaagcccttcggggcaaggagacaggtggtgcatggttgtcgtcagctcgtgtcgtgagatgttaggttaagtcctgcaacgagcgcaacccttgttattagttgctaccattaagttgagcactctaatgagactgcctgggtaaccaggaggaaggtggggatgacgtcaaatcatcatgccccttatgtccagggctacacacgtgctacaatggtaggtacaataagacgcaagaccgtgaggtggagcaaaacttataaaacctatctcagttcggattgtaggctgcaactcgcctacatgaagctggagttgctagtaatcgcgaatcagaatgtcgcggtgaatacgttcccgggccttgtacacaccgcccgtcacaccatgagagctggtaacacccgaagtccgtgaggtaaccgtaaggagccagcggccgaaggtgggattagtgattggggtgaagtcgtaacaaggtagccgtaggagaacctgcggctggatcacctccttt

SEQ ID NO: 32 - DNA, 1509 bp, Clostridium sporogenes
attgagagtttgatcctggctcaggacgaacgctggcggcgtgcttaacacatgcaagtcgagcgatgaagcttccttcgggaagtggattagcggcggacgggtgagtaacacgtgggtaacctgcctcaaagtgggggatagccttccgaaaggaagattaataccgcataacataagagaatcgcatgattttcttatcaaagatttattgctttgagatggacccgcggcgcattagctagttggtaaggtaacggcttaccaaggcaacgatgcgtagccgacctgagagggtgatcggccacattggaactgagacacggtccagactcctacgggaggcagcagtggggaatattgcgcaatgggggaaaccctgacgcagcaacgccgcgtgggtgatgaaggtcttcggattgtaaagccctgttttctgggacgataatgacggtaccagaggaggaagccacggctaactacgtgccagcagccgcggtaatacgtaggtggcgagcgttgtccggatttactgggcgtaaagggtgcgtaggcggatgtttaagtgggatgtgaaatccccgggcttaacctgggggctgcattccaaactggatatctagagtgcaggagaggaaagcggaattcctagtgtagcggtgaaatgcgtagagattaggaagaacaccagtggcgaaggcggctttctggactgtaactgacgctgaggcacgaaagcgtgggtagcaaacaggattagataccctggtagtccacgccgtaaacgatggatactaggtgtagggggtatcaactccccctgtgccgcagttaacacaataagtatcccgcctggggagtacggtcgcaagattaaaactcaaaggaattgacgggggcccgcacaagcagcggagcatgtggtttaattcgaagcaacgcgaagaaccttacctggacttgacatcccttgcatagcctagagataggtgaagcccttcggggcaaggagacaggtggtgcatggttgtcgtcagctcgtgtcgtgagatgttaggttaagtcctgcaacgagcgcaacccttgttattagttgctaccattaagttgagcactctaatgagactgcctgggtaaccaggaggaaggtggggatgacgtcaaatcatcatgccccttatgtccagggctacacacgtgctacaatggtaggtacaataagacgcaagaccgtgaggtggagcaaaacttataaaacctatctcagttcggattgtaggctgcaactcgcctacatgaagctggagttgctagtaatcgcgaatcagaatgtcgcggtgaatacgttcccgggccttgtacacaccgcccgtcacaccatgagagctggtaacacccgaagtccgtgaggtaaccgtaaggagccagcggccgaaggtgggattagtgattggggtgaagtcgtaacaaggtagccgtaggagaacctgcggctggatcacctccttt

SEQ ID NO: 33 - DNA, 1509 bp, Clostridium sporogenes; identical to SEQ ID NO: 32

SEQ ID NO: 34 - DNA, 1509 bp, Clostridium sporogenes; identical to SEQ ID NO: 32

SEQ ID NO: 35 - DNA, 1509 bp, Clostridium sporogenes; identical to SEQ ID NO: 32

SEQ ID NO: 36 - DNA, 1509 bp, Clostridium sporogenes; identical to SEQ ID NO: 32

SEQ ID NO: 37 - DNA, 1509 bp, Clostridium sporogenes; identical to SEQ ID NO: 31

SEQ ID NO: 38 - DNA, 1504 bp, Collinsella tanakaei
cggagagttcgatcctggctcaggatgaacgctggcggcgcgcctaacacatgcaagtcgaacggcacccctcctcggagggaagcgagtggcgaacggctgagtaacacgtggagaacctgccccctcctcggggatagccgcgggaaaccgcgggtaacacccgacgaccccggagccgcgcatgcggcgccgggcaaatctccggagggaggggatggctccgcggcccatcaggtagacggcggggtaacggcccaccgtgccgacaacgggtagccgggttgagagaccgaccggccagattgggactgagacacggcccagactcctacgggaggcagcagtggggaatcttgcgcaatgggggcaaccctgacgcagcgacgccgcgtgcgggacgaaggccttcgggtcgtaaaccgctttcagcagggacgagtcaagacggtacctgcagaagaagccccggctaactacgtgccagcagccgcggtaatacgtagggggcgagcgttatccggattcattgggcgtaaagcgcgcgtaggcggccgcgtaggcggggggtcaaatcccggggctcaaccccggtccgccccccgaaccccgcggctcgggtccggtaggggagggtggaattcccggtgtagcggtggaatgcgcagatatcgggaggaacaccggtggcgaaggcggccctctgggccgagaccgacgctgaggcgcgaaagctgggggagcgaacaggattagataccctggtagtcccagccgtaaacgatggacgctaggtgtggggggacgatccctccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatgtgcgtgaagccggggagacccggtggccgagaggagcgcacacaggtggtgcatggctgtcgtcagctcgtgtcgtgagatgttgggttaagtcccgcaacgagcgcaacccccgccgcatgttgccagcaggtcaggctgggcacccatgcgggaccgccggcgtcaagccggaggagggcggggacgacgtcaagtcatcatgccccttatgccctgggctgcacacgtgctacaatggccggtacagcgggatgcgatggcgcgagccggagcggatccctcaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagccgccggcccaaccttccgaggggggaggcgtcgaaggtgtggagggtgaggggggtgaagtcgtaacaaggtagccgtacgggaacgtgcggctggatcacctccttt

SEQ ID NO: 39 - DNA, 1366 bp, Coprococcus sp HPP0048
aacgagagtttgatcctggctcaggatgaacgctggcggcgtgcttaacacatgcaagtcgaacgaagcacttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcggtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccggcaagtaatgttgcctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgt

SEQ ID NO: 40 - DNA, 1528 bp, Coprococcus sp HPP0048
aacgagagtttgatcctggctcaggatgaacgctggcggcgtgcttaacacatgcaagtcgaacgaagcgcttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccggcaagtaatgttgcctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 41 - DNA, 1366 bp, Coprococcus sp HPP0048
aacgagagtttgatcctggctcaggatgaacgctggcggcgtgcttaacacatgcaagtcgaacgaagcgcttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccggcaagtaatgttgcctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgt

SEQ ID NO: 42 - DNA, 1366 bp, Coprococcus sp HPP0048
aacgagagtttgatcctggctcaggatgaacgctggcggcgtgcttaacacatgcaagtcgaacgaagcacttgaatggaattcttcggaaggaagctagagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccggcaagtaatgttgcctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgt

SEQ ID NO: 43 - DNA, 1434 bp, Coprococcus sp HPP0048
aacgagagtttgatcctggctcaggatgaacgctggcggcgtgcttaacacatgcaagttgaacgaagcgcttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccggcaagtaatgttgcctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaac

SEQ ID NO: 44 - DNA, 1366 bp, Coprococcus sp HPP0048; identical to SEQ ID NO: 42

SEQ ID NO: 45 - DNA, 1530 bp, Flavonifractor plautii
tattgagagtttgatcctggctcaggatgaacgctggcggcgtgcttaacacatgcaagtcgaacggggtgctcatgacggaggattcgtccaatggattgagttacctagtggcggacgggtgagtaacgcgtgaggaacctgccttggagaggggaataacactccgaaaggagtgctaataccgcatgaagcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaggcagagatgcgttaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

SEQ ID NO: 46 - DNA, 1264 bp, Holdemania filiformis
acctaggcgatgatacgtagccgacctgagagggtgaccggccacattgggactgagacacggcccaaactcctacgggaggcagcagtagggaattttcggcaatgggcgaaagcctgaccgagcaacgccgcgtgagtgaagaaggccttcgggttgtaaagctctgttgtgaaggaagaacggctcatacagggaatggtatgggagtgacggtactttaccagaaagccacggctaactacgtgccagcagccgcggtaatacgtaggtggcgagcgttatccggaattattgggcgtaaagggtgcgcaggcggtttgttaagtttaaggtgaaagcgtggggcttaaccccatatagccttagaaactgacagactagagtacaggagagggcaatggaattccatgtgtagcggtaaaatgcgtagatatatggaggaacaccagtggcgaaggcggttgcctggcctgtaactgacgctcatgcacgaaagcgtggggagcaaataggattagataccctagtagtccacgccgtaaacgatgagaactaagtgttggggaaactcagtgctgcagttaacgcaataagttctccgcctggggagtatgcacgcaagtgtgaaactcaaaggaattgacgggggcccgcacaagcggtggagtatgtggtttaattcgacgcaacgcgaagaaccttaccaggtcttgacatcccctgcaaagacatagagatatgttggaggttatcagggagacaggtggtgcatggttgtcgtcagctcgtgtcgtgagatgttgggttaagtcccgcaacgagcgcaacccttgtctttagttgctaacattaagttgaggactctagagagactgccggtgacaaaccggaggaaggtggggatgacgtcaaatcatcatgccccttatgacctgggctacacacgtactacaatggcggatacaacgagaagcaagacagtgatgtggagcaaaactcagaaagtccgtctcagttcggattgaagtctgcaacccgacttcatgaagccggaatcgctagtaatcgcggatcagcatgccgcggtgaatacgttctcgggccttgtacacaccgcccgtcaaaccatgagagttggcaatacccgaagccggtggcctaacctcgcaagaggagggagccgtcgaaggtagggctgatgattggggttaagtcgtaacaaggtatccctacgggaacgtggggatggatcacctccttt

SEQ ID NO: 47 - DNA, 1529 bp, Lachnospiraceae bacterium 2 1 46FAA
aacgagagtttgatcctggctcaggatgaacgctggcggcgtgcttaacacatgcaagtcgaacgaagcgctttcgattgaatcttcggaggaaagaggaagtgactgagtggcggacgggtgagtaacgcgtgggtaacctgcctcatacagggggataacagttagaaatggctgctaataccgcataagcatacagcaccgcatggtgcagtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagtgcaagtctgaagtgaaagcccggggctcaaccccgtgactgctttggaaactgtgcttctagagtgtcggagaggtaagcggaattcctagtgtagcggtgaaatgcgtagatattaggaggaacatcagtggcgaaggcggcttactggacgataactgacgttgaggctcgaaagcgtggggagcaaacaggattagataccctggtagtccacgccgtaaacgatgactactaggtgtcggggtacaaagtacttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctggtcttgacatctccctgaccggacagtaatgtgtcctttctttcgggacagggaagacaggtggtgcatggttgtcgtcagctcgtgtcgtgagatgttgggttaagtcccgcaacgagcgcaacccctatcttcagtagccagcaagtaaagttgggcactctggagagactgccagggataacctggaggaaggtggggacgacgtcaaatcatcatgccccttatgaccagggctacacacgtgctacaatggcgtaaacaaagggaagcaagcatgtgagtgtgagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacctaaccgaaaggaaggagctgccgaaggtgggactgataactggggtgaagtcgtaacaaggtagccgtatcggaaggtgcggctggatcacctccttt

SEQ ID NO: 48 - DNA, 1529 bp, Lachnospiraceae bacterium 2 1 46FAA
aacgagagtttgatcctggctcaggatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcattttcggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 49 - DNA, 1528 bp, Lachnospiraceae bacterium 4 1 37FAA
aacgagagtttgatcctggctcaggatgaacgctggcggcgtgcttaacacatgcaagtcgaacgaagcacttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccggcaagtaatgttgcctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 50 - DNA, 1528 bp, Lachnospiraceae bacterium 6 1 63FAA
tcagagagtttgatcctggctcaggatgaacgctggcggcgtgcttaacacatgcaagtcgagcgaagcacttatcattgactcttcggaagatttgatatttgactgagcggcggacgggtgagtaacgcgtgggtaacctgcctcatacaggggaataacagttagaaatggctgctaatgccgcataagcgcacaggaccgcatggtctggtgtgaaaaactgaggtggtatgagatggacccgcgtctgattaggtagttggtggggtaacggcctaccaagccgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggtgcaaagcagttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatctgcctgaccgttccttaaccggagctttccttcgggacaggcaagacaggtggtgcatggttgtcgtcagctcgtgtcgtgagatgttgggttaagtcccgcaacgagcgcaacccctatccttagtagccagcagtccggctgggcactctagggagactgccggggataacccggaggaaggcggggacgacgtcaaatcatcatgccccttatgatttgggctacacacgtgctacaatggcgtaaacaaagggaagcgaagcggtgacgcttagcaaatctcaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttatggagggagctgccgaaggcgggaccgataactggggtgaagtcgtaacaaggtagccgtatcggaaggtgcggctggatcacctccttt

SEQ ID NO: 51 - DNA, 1528 bp, Lachnospiraceae bacterium 9 1 43BFAA
aacgagagtttgatcctggctcaggatgaacgctggcggcgtgcttaacacatgcaagtcgaacgaagcacttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaatggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccgctctttaatcggagctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 52 - DNA, 1528 bp, Ruminococcus gnavus
aacgagagtttgatcctggctcaggatgaacgctggcggcgtgcttaacacatgcaagtcgagcgaagcacttttgcggatttcttcggattgaagcaattgtgactgagcggcggacgggtgagtaacgcgtgggtaacctgcctcatacagggggataacagttggaaacggctgctaataccgcataagcgcacagtaccgcatggtaccgtgtgaaaaactccggtggtatgagatggacccgcgtctgattagctagttggtggggtaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccccctgaccgctctttaatcggagctttccttcgggacaggggagacaggtggtgcatggttgtcgtcagctcgtgtcgtgagatgttgggttaagtcccgcaacgagcgcaacccctatctttagtagccagcatttcggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 53 - DNA, 1528 bp, Ruminococcus gnavus
aacgagagtttgatcctggctcaggatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactgccagggataacctggaggaaggtggggatgacgtcaaatcatcatgccccttatgatcagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 54 - DNA, 1528 bp, Ruminococcus gnavus
aacgagagtttgatcctggctcaggatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccccctgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactgccagggataacctggaggaaggtggggatgacgtcaaatcatcatgccccttatgatcagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 55 - DNA, 1242 bp, Ruminococcus gnavus
  misc_feature (1)..(1): n is a, c, g, or t
ncgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggaactttccttcgggacagaggagacaggtggtgcatggttgtcgtcagctcgtgtcgtgagatgttgggttaagtcccgcaacgagcgcaacccctatctttagtagccagcattttggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 56 - DNA, 1528 bp, Ruminococcus gnavus
aacgagagtttgatcctggctcaggatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcatttcggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 57 - DNA, 1528 bp, Ruminococcus gnavus
aacgagagtttgatcctggctcaggatgaacgctggcggcgtgcttaacacatgcaagtcgagcgaagcgcttttgcggatttcttcggattgaagcaactgtgactgagcggcggacgggtgagtaacgcgtgggtaacctgcctcatacagggggataacagttggaaacggctgctaataccgcataagcgcacagtaccgcatggtaccgtgtgaaaaactccggtggtatgagatggacccgcgtctgattagctagttggtggggtaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggtaagcggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctatgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactgccagggataacctggaggaaggtggggatgacgtcaaatcatcatgccccttatgaccagggctacacacgtgctacaatggcgtaaacaaagggaagcgaacctgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 58 - DNA, 1529 bp, Ruminococcus torques
aacgagagtttgatcctggctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtggccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactgggggtgaagtcgtaacaaggtagccgtatcggaaggtgcggctggatcacctccttt

SEQ ID NO: 59 - DNA, 1529 bp, Ruminococcus torques
aacgagagtttgatcctggctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtccccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctccttt

SEQ ID NO: 60 - DNA, 1056 bp, Ruminococcus torques
aacgagagtttgatcctggctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtgggagcatgtggtttaattcgaagcaacgcgaagaaccttacctgctcttgacatcccgctgaccggacggtaatgcgtccttcccttcgggcagcggagacaggtggtgcatggttgtcgtcagct

SEQ ID NO: 61 - DNA, 1529 bp, Ruminococcus torques; identical to SEQ ID NO: 58

SEQ ID NO: 62 - DNA, 1177 bp, Ruminococcus torques
aacgagagtttgatcctggctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctggttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctgggggagtacgttcgcaagaatgaaactcaaaggaattgacgggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcacgagcgcaaccctatctttagtagcagcggccaggccgggcactctaaaagagactgccggggataatcccgggaggaaggtggggat

SEQ ID NO: 63 - DNA, 1528 bp, Ruminococcus torques
tacgagagtttgatcctggctcaggatgaacgcgggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtggccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctccttt

SEQ ID NO: 64 - DNA, 1342 bp, Staphylococcus aureus
aagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctt

SEQ ID NO: 65 - DNA, 1561 bp, Veillonella parvula
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggatagatgaaaggtggcctctacatgtaagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaacagttagaaggattgacggtaccggaatagaaagccacggctaactacgtgccagcagccgcggtaatacgtaggtggcaagcgttgtccggaattattgggcgtaaagcgcgcgcaggcggatcagtcagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacgtaatggtgggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctccttt

SEQ ID NO: 66 - DNA, 1359 bp, Clostridium ghonni
tcgagcgaacccttcggggtgagcggcggacgggtgagtaacgcgtgggtaacctgccctgtacacacggataacataccgaaaggtatgctaatacgggataacatatgagagtcgcatggcttttgtatcaaagctccggcggtacaggatggacccgcgtctgattagctagttggtaaggtaacggcttaccaaggcaacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggcggtctttcaagccagaagtgaaaggctacggctcaaccgtagtaagcttttggaactgtaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccattgacctctccctaatcggagatttcccttcggggacagtggtgacaggtggtgcatggctgtcgtcagctcgtgtcgtgagatgttggggtaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgtgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttgggggcgcccgaagccggttagctaaccttttaggaagcg

SEQ ID NO: 67 - DNA, 1381 bp, Bacteroides ovatus
  misc_feature (25)..(25): N is A, C, T, G, unknown, or other
tcgaggggcagcattttagtttgcnttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggagccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaattggctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaa

SEQ ID NO: 68 - DNA, 1261 bp, Bacteroides stercoris
atggtatagttttcccgcatgggataattattaaagaatttcggttgtcgatggggatgcgttccattaggcagttggcggggtaacggcccaccaaacctacgatggataggggttctgagaggaaggtcccccacattggaactgagacacggtccaaactcatacgggaggcagcagtgaggaatattggtcaatggacgagagtctgaaccagccaagtagcgtgaaggatgactgccctatgggttgtaaacttcttttatacgggaataaagttagccacgtgtggttttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacggtcagcggccaagcgaaagcattaagtattccacctggggagtacgccggcagcggtgaaactcaaaggaattgacgggggcccgcacaagcggaggaacatgtggtttaattcgatgatacgcgaggaaccttacccgggcttaaattgcaactgacggaatcggaaacggttctttcttcggacagttgtgaaggtgctgcatggttgtcgtcagctcgtgccgtgaggtgtcggcttaagtgccataacgagcgcaacccttacgggtagttaccatcaggttatgctggggactctacccggactgccgtcgtaagatgtgaggaaggtggggatgacgtcaaatcagcacggcccttacgtccggggctacacacgtgttacaatggggggtacagaaggcagctacacggcgacgtggtgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcga

SEQ ID NO: 69 - DNA, 999 bp, Eggerthella lenta
  misc_feature (33)..(36): Ns are each independently A, C, T, G, unknown or other
  misc_feature (57)..(57): Ns are each independently A, C, T, G, unknown or other
  misc_feature (119)..(119): Ns are each independently A, C, T, G, unknown or other
  misc_feature (788)..(788): Ns are each independently A, C, T, G, unknown or other
  misc_feature (848)..(848): Ns are each independently A, C, T, G, unknown or other
  misc_feature (860)..(860): Ns are each independently A, C, T, G, unknown or other
  misc_feature (877)..(877): Ns are each independently A, C, T, G, unknown or other
  misc_feature (877)..(877): Ns are each independently A, C, T, G, unknown or other
  misc_feature (887)..(887): Ns are each independently A, C, T, G, unknown or other
  misc_feature (891)..(891): Ns are each independently A, C, T, G, unknown or other
  misc_feature (899)..(899): Ns are each independently A, C, T, G, unknown or other
  misc_feature (933)..(944): Ns are each independently A, C, T, G, unknown or other
  misc_feature (961)..(961): Ns are each independently A, C, T, G, unknown or other
  misc_feature (963)..(963): Ns are each independently A, C, T, G, unknown or other
  misc_feature (978)..(978): Ns are each independently A, C, T, G, unknown or other
  misc_feature (980)..(980): Ns are each independently A, C, T, G, unknown or other
  misc_feature (983)..(983): Ns are each independently A, C, T, G, unknown or other
cctcgggcggacatgaagtggcgaacgggtgannnncacgtgaccaacctgcccccngctccgggacaaccttgggaaaccgaggctaataccggatactccctcccctgctcctgggngggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggncgcaaggctaaaactcaaggaattgacgggggcccgcacaagcagcggagcatgtggctnaattcgaagcancgcgaagaaccttaccngggcttgacntggnacgtgaanccggggaaacccggtggccgagaggagcgtccgnnagtggtgcatggctgtcgtcagctcgngncgtgagatgttgggntnagntcccgcaacgagcgca

SEQ ID NO: 70 - DNA, 753 bp, Eggerthella sp. YY7918
  misc_feature (122)..(122): Ns are each independently A, C, T, G, unknown or other
  misc_feature (197)..(197): Ns are each independently A, C, T, G, unknown or other
  misc_feature (341)..(341): Ns are each independently A, C, T, G, unknown or other
  misc_feature (431)..(431): Ns are each independently A, C, T, G, unknown or other
  misc_feature (508)..(508): Ns are each independently A, C, T, G, unknown or other
  misc_feature (602)..(602): Ns are each independently A, C, T, G, unknown or other
  misc_feature (620)..(621): Ns are each independently A, C, T, G, unknown or other
  misc_feature (623)..(623): Ns are each independently A, C, T, G, unknown or other
  misc_feature (627)..(627): Ns are each independently A, C, T, G, unknown or other
  misc_feature (670)..(670): Ns are each independently A, C, T, G, unknown or other
  misc_feature (676)..(676): Ns are each independently A, C, T, G, unknown or other
  misc_feature (709)..(709): Ns are each independently A, C, T, G, unknown or other
  misc_feature (739)..(739): Ns are each independently A, C, T, G, unknown or other
ctacccttctcttcgggacaaccttgggaaaccgaggctaataccgaatactccggtgagtgcgcatgcacacgccgggaaagctttcgcggagaaggatggggtcgcggcccatcaggtanacggcggggtagcggcccaccgtgcttttgacgggtagccgggttgagagaccgaccggccacattgggactganatacggcccagactcctacgggaggcagcagtggggaattttgcgcaatgggggaaaccctgacgcagcaccgccgcgtgcgggatgaaggccttcgggtcgtaaaccgctttcagcagggaagaaattcgacggtacctgcanaagaagccccggctaactacgtgccagcagccgcggtaatacgtagggggcgagcgttatccggattcattgggcgtaaagcgcgcgtangcggccgcctaagcgggacctctaaccttggggctcaacctcaagccgggttccgaactgggtggctcgagtttggnagaggaagatggaattcccggtgtagcggtggaatgcgcagatatcgggaagaacaccgatggcgaaggcagtcttctgggctcataactgacnctgaagcgcgaaagctanngnagcnaacaggattagataccctggtagtcctagccgtaaacgatggncactangtgtggggagatactctttccgtgccgaagcanacgcattaagtgccccgcctggggagtacngccgcaaggctaaa

SEQ ID NO: 71 - DNA, 1346 bp, Eggerthella sp. 1_3_56FAA
  misc_feature (123)..(123): Ns are each independently A, C, T, G, unknown or other
  misc_feature (1329)..(1329): Ns are each independently A, C, T, G, unknown or other
accgccctcgggcggacatgaagtggcgaacgggtgagtaacacgtgaccaacctgcccccctctccgggacaaccttgggaaaccgaggctaataccggatactccctcccctgctcctggnggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcaggcctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttagttcgaagcaacgcgaagaacctcaccaggcctgatcccgaactggaagccggggaaacccggtggccgagaggaacgtccgcaggtggtgcatggctgtcgtcagctcgtgtcgtgacatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccntccgaacccgcaagggg

SEQ ID NO: 72 - DNA, 1430 bp, Eggerthella sp. HGA1
gaacgctggcggcgtgcctaacacatgcaagtcgaacgatgaaaccgccctcgggcggacatgaagtggcgaacgggtgagtaacacgtgaccaacctgcccccctctccgggacaaccttgggaaaccgaggctaataccggatactccctcccctgctcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggcaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaaggtgtggagggtaaggggggtgaa

SEQ ID NO: 73 - protein, 417 aa, Clostridium sporogenes
MKFWRKYTQQEMDEKITESLEKTLNYDNTKTIGIPGTKLDDTVFYDDHSFVKHSPYLRTFIQNPNHIGCHTYDKADILFGGTFDIERELIQLLAIDVLNGNDEEFDGYVTQGGTEANIQAMWVYRNYFKKERKAKHEEIAIITSADTHYSAYKGSDLLNIDIIKVPVDFYSRKIQENTLDSIVKEAKEIGKKYFIVISNMGTTMFGSVDDPDLYANIFDKYNLEYKIHVDGAFGGFIYPIDNKECKTDFSNKNVSSITLDGHKMLQAPYGTGIFVSRKNLIHNTLTKEATYIENLDVTLSGSRSGSNAVAIWMVLASYGPYGWMEKINKLRNRTKWLCKQLNDMRIKYYKEDSMNIVTIEEQYVNKEIAEKYFLVPEVHNPTNNWYKIVVMEHVELDILNSLVYDLRKFNKEHLKAM

SEQ ID NO: 74 - protein, 490 aa, Ruminococcus gnavus
MSQVIKKKRNTFMIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTENPGGVFVSGGSMANITALTAARDNKLTDINLHLGTAYISDQTHSSVAKGLRIIGITDSRIRRIPTNSHFQMDTTKLEEAIETDKKSGYIPFVVIGTAGTTNTGSIDPLTEISALCKKHDMWFHIDGAYGASVLLSPKYKSLLTGTGLADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNPKKDWEIVSPAQMAMINFRYAPKDLTKEEQDILNEKISHRILESGYAAIFTTVLNGKTVLRICAIHPEATQEDMQHTIDLLDQYGREIYTEMKKA

SEQ ID NO: 75 - protein, 490 aa, Ruminococcus gnavus; identical to SEQ ID NO: 74